(19) United States
(12) Patent Application Publication
(10) Pub. No.: US 2024/0000865 A1
(43) Pub. Date: Jan. 4, 2024

(54) BACILLUS SUBTILIS STRAIN WITH STRONG INHIBITION OF ENTEROPATHOGENIC AND FOODBORNE PATHOGENIC BACTERIA

(71) Applicants: UNIVERZA V LJUUBLJANI, Ljubljana (SI); lowa State University Research Foundation, Inc., Ames, IA (US)

(72) Inventors: Ines Mandic Mulec, Ljubljana (SI); Katarina Simunovic, Ljubljana (SI); Polonca Stefanic, Ljubljana (SI); Andi Erega, Ljubljana (SI); Sonja Smole Mozina, Ljubljana (SI); Anja Klancnik, Ljubljana (SI); Qijing Zhang, Ames, IA (US); Orhan Sahin, Ames, IA (US)

(21) Appl. No.: 18/038,032

(22) PCT Filed: Nov. 22, 2021
(86) PCT No.: PCT/EP2021/082499

§ 371 Date: May 22, 2023

Related U.S. Application Data
(60) Provisional application No. 63/117,215, filed on Nov. 23, 2020.

(30) Foreign Application Priority Data
Jan. 18, 2021 (LU) LU102419
Jan. 18, 2021 (LU) LU102420

(51) Int. Cl. A61K 35/742; A23K 10/18; A23K 50/75; A23K 50/60; C12N 1/20; A61K 9/00; A61P 31/04

(52) U.S. Cl. CPC A61K 35/742; A23K 10/18; A23K 50/75; A23K 50/60; C12N 1/205; A61K 9/0056; A61P 31/04; C12R 2001/125

(57) ABSTRACT

The present invention generally relates to the field of microbiology, and more specifically to the Bacillus subtilis strain PS-216, which has been shown to have strong inhibitory activity against enteropathogenic and/or foodborne pathogenic bacteria, such as Campylobacter jejuni. More particularly, the present invention provides to new methods and uses of the Bacillus subtilis strain PS-216. The present invention also provides feed or food compositions and probiotic compositions comprising this strain.

### FIELD OF THE INVENTION

The present invention generally relates to the field of microbiology, and more specifically to the Bacillus subtilis strain PS-216, which has been shown to have strong inhibitory activity against enteropathogenic and/or foodborne pathogenic bacteria, such as Campylobacter jejuni. More particularly, the present invention pertains to new methods and uses of the Bacillus subtilis strain PS-216. The present invention also pertains to feed or food compositions and probiotic compositions comprising this strain.

### BACKGROUND OF THE INVENTION

Enteric and foodborne pathogens present a major healthcare and economic burden. Amongst the major pathogens causing diarrhea are Listeria spp., Staphylococcus spp., Salmonella spp., E. coli, and Campylobacter spp. (EFSA and ECDC, 2018).

Campylobacteriosis is the most frequently reported bacterial foodborne infection both in the European Union (EU) and the United States (US), and its major cause is Campylobacter jejuni. The disease symptoms include, diarrhoea, fever and cramps, which can lead to the development of Guillain-Barre syndrome, a severe neurological condition (Kaakoush et al., 2015; Nachamkin et al., 1998; EFSA and ECDC 2018). Campylobacter infections are a major burden on health care industries and national economies, as the estimated total annual cost of the disease and its consequences is €2.4 billion (BIOHAZ, 2011; EFSA and ECDC, 2018). New and better interventions are needed to tackle this pathogen.

Most of C. jejuni infections have been associated with poultry meat and the poultry industry, which is also a major source of antibiotic resistant C. jejuni strains, as more than 50% of isolates from poultry are now resistant to at least one antibiotic. This presents a high risk of resistant C. jejuni spreading through the food chain (EFSA and ECDC, 2019).

Because C. jejuni is a common avian commensal, efforts are being made to tackle the pathogen at its primary source, the chicken reservoir. Once Campylobacter has been introduced into a farm, its fast spread is imminent (Berndtson et al., 1996), thus are effective control measures of great importance. These can include pre-harvest measures (biosecurity and hygiene measures) for prevention of Campylobacter entrance onto a farm and limitation of its spread, and post-harvest measures (freezing, hot-water treatment, irradiation, and chemical decontamination) for reduction of Campylobacter after slaughter (Sahin et al., 2015).

The reduction of Campylobacter in the chicken intestine by even 1 log₁₀ CFU can reduce the public health risk by 50% to 90%, thus is the reduction of Campylobacter in the poultry production the best starting point for improvement (BIOHAZ, 2011; Meunier et al. 2016; Dogan et al. 2019).

Probiotics can be used as a pre-harvest measure for pathogen control on the poultry farm (Alagawany et al., 2018; Sahin et al., 2015). Probiotics can have beneficial effects on poultry, such as growth promotion, immunomodulation, and inhibition of pathogens. Modes of action of probiotic bacteria against pathogens can include production of organic acids and antibacterial substances, competitive exclusion of pathogens, modulation of the host immune system, and others. Amongst bacteria studied and used in poultry pharming as probiotics, Lactobacillus spp., Bifidobacterium spp., Bacillus spp. Streptococcus spp. and Enterococcus spp. can be found (Alagawany et al., 2018; Hong et al., 2005; Lutful Kabir, 2009).

Bacillus subtilis strains as poultry probiotics can be found in commercial formulations, such as GalliPro (Chr Hansen), Calsporin (ORFFA) and Alterion (Novozymes). Different B. subtilis strains, either alone or in combination with other bacteria, have shown to improve feed conversion and body weight in chickens, reduce lesions caused by Clostridium perfringens, prolong intestinal villi in necrotic enteritis, modulate microbiota to improve Lactobacillus counts in intestine, and lower the count of pathogens such as C. perfringens, Escherichia coli, Salmonella enteritidis and others, in chickens (Fritts et al., 2000; Hayashi et al., 2018; Hmani et al., 2017; Jayaraman et al., 2013; Park et al., 2017; Zhou et al., 2015). Different B. subtilis strains have shown some promising results with C. jejuni reduction, although this anti-Campylobacter effect is strains specific (Saint-Cyr et al., 2016). Not all B. subtilis treatment will result in lowered C. jejuni numbers in the chicken intestine making the proper selection of an anti-CampylobacterB. subtilis strain very important.

Accordingly, it is one object of the present invention to provide a Bacillus subtilis strain having improved anti-Campylobacter activity.

### SUMMARY OF THE INVENTION

The present invention is based on the surprising finding that Bacillus subtilis strain PS-216 has the ability to greatly reduce C. jejuni colonization in broilers compared to other B. subtilis strains. Moreover, it has been shown that the treatment of broilers with B. subtilis PS-216 results in an increased weight of broilers. The present inventors have further found that B. subtilis PS-216 inhibits the formation of biofilms of C. jejuni on abiotic surfaces and is able to disintegrate preestablished C. jejuni biofilm.

In general terms, the present invention thus provides methods and uses of the B. subtilis strain PS-216, or a derivative thereof having all of the identifying characteristics of B. subtilis PS-216, for inhibiting, reducing or preventing the colonization of an enteropathogenic and/or foodborne pathogenic bacterium, such as C. jejuni, in a subject, such as a non-human animal.

The present invention further provides methods and uses of the B. subtilis strain PS-216, or a derivative thereof having all of the identifying characteristics of B. subtilis PS-216, for improving the health status, in particular the gut health status, of a subject, such as a non-human animal.

The present invention further provides methods and uses of the B. subtilis strain PS-216, or a derivative thereof having all of the identifying characteristics of B. subtilis PS-216, for enhancing the growth of a non-human animal, such as poultry animal.

The present invention further provides methods and uses of the B. subtilis strain PS-216, or a derivative thereof having all of the identifying characteristics of B. subtilis PS-216, for increasing the weight of a non-human animal, such as poultry animal.

The present invention further provides a feed or food composition containing the B. subtilis strain PS-216, or a derivative thereof having all of the identifying characteristics of B. subtilis PS-216, and at least one further feed or food ingredient.

The present invention further provides a probiotic composition comprising the B. subtilis strain PS-216, or a derivative thereof having all of the identifying characteristics of B. subtilis PS-216, and at least one excipient, such as at least one pharmaceutically or agriculturally acceptable excipient (e.g., carrier). The probiotic composition is particularly useful in the treatment and/or prevention of a microbial infection or colonization by an enteropathogenic and/or foodborne pathogenic bacterium, such as C. jejuni, in a subject, such as a non-human animal.

The present invention further provides the use of the B. subtilis strain PS-216, or a derivative thereof having all of the identifying characteristics of B. subtilis PS-216, as a probiotic ingredient (DFM) in a feed or food product.

The present invention further provides methods and uses of the B. subtilis strain PS-216, or a derivative thereof having all of the identifying characteristics of B. subtilis PS-216, for inhibiting, reducing or preventing biofilm formation or buildup of an enteropathogenic and/or foodborne pathogenic bacterium, such as C. jejuni, on an abiotic surface.

The present invention may be further summarized by the following items:

1. A method for inhibiting, reducing or preventing the colonization of an enteropathogenic and/or foodborne pathogenic bacterium, such as C. jejuni, in a subject comprising administering an effective amount of the Bacillus subtilis strain PS-216, or a derivative thereof having all of the identifying characteristics of B. subtilis PS-216, to the subject.

2. The method according to item 1, wherein the enteropathogenic and/or foodborne pathogenic bacterium is a gram-negative bacterium.

3. The method according to item 1 or 2, wherein the enteropathogenic and/or foodborne pathogenic bacterium is a bacterium of the family Campylobacteraceae or Enterobacteriaceae.

4. The method according to any one of items 1 to 3, wherein the enteropathogenic and/or foodborne pathogenic bacterium is a bacterium of the family Campylobacteraceae.

5. The method according to any one of items 1 to 3, wherein the enteropathogenic and/or foodborne pathogenic bacterium is a bacterium of the genus Campylobacter.

6. The method according to any one of items 1 to 3, wherein the enteropathogenic bacterium is a bacterium selected from the group consisting of: C. jejuni, C. coli, C. concisus, C. fetus, C. hyoilei, C. helveticus, C. hyointestinalis, C. lari, C. mucosalis, C. sputorum and C. upsaliensis.

7. The method according to any one of items 1 to 3, wherein the enteropathogenic and/or foodborne pathogenic bacterium is a bacterium of the species C. jejuni or C. coli.

8. The method according to any one of items 1 to 3, wherein the enteropathogenic and/or foodborne pathogenic bacterium is a bacterium of the species C. jejuni.

9. The method according to any one of items 1 to 3, wherein the enteropathogenic and/or foodborne pathogenic bacterium is a bacterium of the family Enterobacteriaceae.

10. The method according to any one of items 1 to 3, wherein the enteropathogenic and/or foodborne pathogenic bacterium is a bacterium of the genus Listeria, Escherichia, Yersinia., Vibrio, Salmonella or Shigella.

11. The method according to any one of items 1 to 3, wherein the enteropathogenic bacterium is a bacterium selected from the group consisting of: Listeria monocytogenes, Yersinia enterocolitica, Vibrio parachaemoliticus, Escherichia coli, Salmonella enterica, including Salmonella Enteritidis and Salmonella Infantis, and Shigella dysenteriae.

12. A method for improving the health status, such as the gut health status, of a subject, such as poultry animal, comprising administering an effective amount of the B. subtilis strain PS-216, or a derivative thereof having all of the identifying characteristics of B. subtilis PS-216, to the subject.

13. A method for enhancing the growth of a non-human animal, such as poultry animal, comprising administering an effective amount of the B. subtilis strain PS-216, or a derivative thereof having all of the identifying characteristics of B. subtilis PS-216, to the non-human animal.

14. A method for increasing the weight of a non-human animal, such as poultry animal, comprising administering an effective amount of the B. subtilis strain PS-216, or a derivative thereof having all of the identifying characteristics of B. subtilis PS-216, to the non-human animal.

15. The method according to any one of items 1 to 12, wherein the subject is a non-human animal.

16. The method according to any one of items 13 to 15, wherein the non-human animal is a productive animal.

17. The method according to item 16, wherein the productive animal is selected from the group consisting of pig, chicken, duck, goose, turkey, cow, sheep and goat.

18. The method according to any one of items 13 to 15, wherein the non-human animal is a poultry animal.

19. The method according to item 18, wherein the poultry animal is a productive poultry animal.

20. The method according to item 18 or 19, wherein the poultry animal is selected from the group consisting of: chicken, duck, goose, turkey, guinea fowl and pigeon.

21. The method according to item 18 or 19, wherein the poultry animal is a chicken.

22. The method according to any one of items 13 to 21, wherein the non-human animal is under four weeks old.

23. The method according to any one of items 13 to 22, wherein the non-human animal is between 1 and 21 days old.

24. The method according to any one of items 13 to 23, wherein the non-human animal is between 1 and 14 days old.

25. The method according to any one of items 13 to 24, wherein the non-human animal is between 1 and 7 days old.

26. The method according to any one of items 13 to 23, wherein the non-human animal is between 13 and 21 days old.

27. The method according to any one of items 1 to 12, wherein the subject is a human.

28. The method according to any one of items 1 to 27, wherein the strain is administered in the form of spores, cells, vegetative cells or a dried cell mass.

29. The method according to any one of items 1 to 28, wherein the strain is administered in the form of spores.

30. The method according to any one of items 1 to 29, wherein the strain is administered at a bacterial load of at least about 1×10² colony forming units (CFU).

31. The method according to any one of items 1 to 30, wherein the strain is administered at a bacterial load in a range of from about 1×10² to about 1×10¹⁴ colony forming units.

32. The method according to any one of items 1 to 31, wherein the strain is administered at a bacterial load in a range of from about 1×10⁴ to about 1×10¹⁰ colony forming units.

33. The method according to any one of items 1 to 32, wherein the strain is administered at a bacterial load in a range of from about 1×10⁴ to about 1×10⁹ colony forming units.

34. The method according to any one of items 1 to 33, wherein the strain is administered at a bacterial load in a range of from about 1×10⁴ to about 1×10⁸ colony forming units.

35. The method according to any one of items 1 to 34, wherein the strain is administered in an aqueous solution.

36. The method according to any one of items 1 to 34, wherein the strain is administered as a dry feed.

37. The method according to any one of items 1 to 34, wherein the strain is administered the feed composition of the present invention.

38. The method according to any one of items 1 to 37, wherein the strain is administered perorally.

39. The method according to any one of items 1 to 35, wherein the strain is administered by aerosol.

40. The method according to any one of items 1 to 35, wherein the strain is administered in a water solution containing from about 1×10⁴ to about 1×10⁸ CFU/mL, such as about 2.5×10⁶ CFU/mL, B. subtilis PS-216 spores as drinking water.

41. B. subtilis strain PS-216, or a derivative thereof having all of the identifying characteristics of B. subtilis PS-216, for use in inhibiting, reducing or preventing the colonization of an enteropathogenic and/or foodborne pathogenic bacterium, such as C. jejuni, in a subject.

42. B. subtilis strain for use according to item 41, wherein the enteropathogenic and/or foodborne pathogenic bacterium is a gram-negative bacterium.

43. B. subtilis strain for use according to item 41 or 42, wherein the enteropathogenic and/or foodborne pathogenic bacterium is a bacterium of the family Campylobacteraceae or Enterobacteriaceae.

44. B. subtilis strain for use according to any one of items 41 to 43, wherein the enteropathogenic and/or foodborne pathogenic bacterium is a bacterium of the family Campylobacteraceae.

45. B. subtilis strain for use according to any one of items 41 to 43, wherein the enteropathogenic and/or foodborne pathogenic bacterium is a bacterium of the genus Campylobacter.

46. B. subtilis strain for use according to any one of items 41 to 43, wherein the enteropathogenic and/or foodborne pathogenic bacterium is a bacterium selected from the group consisting of: C. jejuni, C. coli, C. concisus, C. fetus, C. hyoilei, C. helveticus, C. hyointestinalis, C. lari, C. mucosalis, C. sputorum and C. upsaliensis.

47. B. subtilis strain for use according to any one of items 41 to 43, wherein the enteropathogenic and/or foodborne pathogenic bacterium is a bacterium of the species C. jejuni or C. coli.

48. B. subtilis strain for use according to any one of items 41 to 43, wherein the enteropathogenic and/or foodborne pathogenic bacterium is a bacterium of the species C. jejuni.

49. B. subtilis strain for use according to any one of items 41 to 43, wherein the enteropathogenic and/or foodborne pathogenic bacterium is a bacterium of the family Enterobacteriaceae.

50. B. subtilis strain for use according to any one of items 41 to 43, wherein the enteropathogenic and/or foodborne pathogenic bacterium is a bacterium of the genus Listeria, Escherichia, Yersinia, Vibrio, Salmonella or Shigella.

51. B. subtilis strain for use according to any one of items 41 to 43, wherein the enteropathogenic and/or foodborne pathogenic bacterium is a bacterium selected from the group consisting of: Listeria monocytogenes, Yersinia enterocolitica, Vibrio parachaemoliticus, Escherichia coli, Salmonella enterica, including Salmonella Enteritidis and Salmonella Infantis, and Shigella dysenteriae.

52. B. subtilis strain PS-216, or a derivative thereof having all of the identifying characteristics of B. subtilis PS-216, for use in improving the health status, such as the gut health status, of a subject.

53. B. subtilis strain PS-216, or a derivative thereof having all of the identifying characteristics of B. subtilis PS-216, for use in enhancing the growth of a non-human animal.

54. B. subtilis strain PS-216, or a derivative thereof having all of the identifying characteristics of B. subtilis PS-216, for use in increasing the weight of a non-human animal.

55. B. subtilis strain for use according to any one of items 41 to 52, wherein the subject is a non-human animal.

56. B. subtilis strain for use according to any one of items 53 to 55, wherein the non-human animal is a productive animal.

57. B. subtilis strain for use according to item 56, wherein the productive animal is selected from the group consisting of pig, chicken, duck, goose, turkey, cow, sheep and goat.

58. B. subtilis strain for use according to any one of items 53 to 55, wherein the non-human animal is a poultry animal.

59. B. subtilis strain for use according to item 58, wherein the poultry animal is a productive poultry animal.

60. B. subtilis strain for use according to item 58 or 59, wherein the poultry animal is selected from the group consisting of: chicken, duck, goose, turkey, guinea fowl and pigeon.

61. B. subtilis strain for use according to item 58 or 59, wherein the poultry animal is a chicken.

62. B. subtilis strain for use according to any one of items 53 to 61, wherein the non-human animal is under four weeks old.

63. B. subtilis strain for use according to any one of items 53 to 62, wherein the non-human animal is between 1 and 21 days old.

64. B. subtilis strain for use according to any one of items 53 to 63, wherein the non-human animal is between 1 and 14 days old.

65. B. subtilis strain for use according to any one of items 53 to 64, wherein the non-human animal is between 1 and 7 days old.

66. B. subtilis strain for use according to any one of items 53 to 63, wherein the non-human animal is between 13 and 21 days old.

67. B. subtilis strain for use according to item 41 to 52, wherein the subject is a human.

68. B. subtilis strain for use according to any one of items 41 to 67, wherein the strain is in the form of spores, cells, vegetative cells or a dried cell mass.

69. B. subtilis strain for use according to any one of items 41 to 68, wherein the strain is administered in the form of spores.

70 B. subtilis strain for use according to any one of items 41 to 69, wherein the strain is administered at a bacterial load of at least about 1×10² colony forming units (CFU).

71. B. subtilis strain for use according to any one of items 41 to 70, wherein the strain is administered at a bacterial load in a range of from about 1×10² to about 1×10¹⁴ colony forming units.

72. B. subtilis strain for use according to any one of items 41 to 71, wherein the strain is administered at a bacterial load in a range of from about 1×10⁴ to about 1×10¹⁰ colony forming units.

73. B. subtilis strain for use according to any one of items 41 to 72, wherein the strain is administered at a bacterial load in a range of from about 1×10⁴ to about 1×10⁹ colony forming units.

74. B. subtilis strain for use according to any one of items 41 to 73, wherein the strain is administered at a bacterial load in a range of from about 1×10⁴ to about 1×10⁸ colony forming units.

75. B. subtilis strain for use according to any one of items 41 to 74, wherein the strain is administered in an aqueous solution.

76. B. subtilis strain for use according to any one of items 41 to 74, wherein the strain is administered as a dry feed.

77. B. subtilis strain for use according to any one of items 41 to 74, wherein the strain is administered as part of the feed composition of the present invention.

78. B. subtilis strain for use according to any one of items 41 to 77, wherein the strain is administered perorally.

79. B. subtilis strain for use according to any one of items 41 to 75, wherein the strain is administered by aerosol.

80. B. subtilis strain for use according to any one of items 41 to 75, wherein the strain is administered in a water solution containing from about 1×10⁴ to about 1×10⁸ CFU/mL, such as about 2.5×10⁶ CFU/mL, B. subtilis PS-216 spores as drinking water.

81. A feed or food composition containing the B. subtilis strain PS-216, or a derivative thereof having all of the identifying characteristics of B. subtilis PS-216, and at least one further feed or food ingredient.

82. The feed or food composition according to item 81, wherein the feed or food ingredient is selected from proteins, carbohydrates, fats, fibers, further probiotics, prebiotics, enzymes, vitamins, minerals, amino acids, and combinations thereof.

83. The feed or food composition according to item 81, comprising at least one vitamin.

84. The feed or food composition according to item 83, wherein the at least one vitamin is selected from the group consisting of Vitamin A, Vitamin E and a combination thereof.

85. The feed or food composition according to any one of items 81 to 84, comprising at least one mineral.

86. The feed or food composition according to item 85, wherein the at least one mineral is selected from the group consisting of calcium, phosphorus, manganese, sodium (such as in the form of sodium chloride) and combinations thereof.

87. The feed or food composition according to any one of items 81 to 86, comprising at least one amino acid.

88. The feed or food composition according to item 87, wherein the at least one amino acid is a natural amino acid.

89. The feed or food composition according to item 87 or 88, wherein the at least one amino acid is an essential amino acid.

90. The feed or food composition according to any one of items 87 to 89, wherein the at least one amino acid is selected from the group consisting of histidine, isoleucine, lysine, methionine, phenylalanine, threonine, tryptophan, valine and combinations thereof.

91. The feed or food composition according to any one of items 87 to 89, wherein the at least one amino acid is selected from the group consisting of lysine, methionine and a combination thereof.

92. The feed or food composition according to any one of items 81 to 91, comprising at least one fat, such as crude fat.

93. The feed or food composition according to any one of items 81 to 92, comprising at least one fiber, such as crude fiber.

94. The feed or food composition according to any one of items 81 to 93, comprising at least one protein, such as crude protein.

95. The feed or food composition according to any one of items 81 to 94, comprising at least one enzyme.

96. The feed or food composition according to item 95, wherein the at least one enzyme is selected from the group consisting of selected from phytases, xylanases, galactanases, galactosidases, proteases, phospholipases, lysophospholipases, amylases, lysozymes, glucanases, glucoamylases, cellulases, pectinases, and combinations thereof.

97. The feed or food composition according to item 95, wherein the at least one enzyme is a phytase.

98. The feed or food composition according to any one of items 81 to 97, wherein the strain is in the form of spores, cells, vegetative cells or a dried cell mass.

99. The feed or food composition according to any one of items 81 to 98, wherein the strain is in the form of spores.

100. The feed or food composition according to any one of items 81 to 99, containing at least about 1×10³ colony forming units (CFU) of the strain/kg of composition.

101. The feed or food composition according to any one of items 81 to 100, containing from about 1×10³ to about 1×10¹⁴ colony forming units of the strain/kg of composition.

102. A probiotic composition comprising the B. subtilis strain PS-216, or a derivative thereof having all of the identifying characteristics of B. subtilis PS-216, and at least one excipient, such as at least one pharmaceutically or agriculturally acceptable excipient.

103. The probiotic composition according to item 102, wherein the strain is in the form of spores, cells, vegetative cells or a dried cell mass.

104. The probiotic composition according to item 102 or 103, wherein the strain is in the form of spores.

105. The probiotic composition according to any one of items 102 to 104, containing at least about 1×10³ colony forming units (CFU) of the strain/kg of composition.

106. The probiotic composition according to any one of items 102 to 105, containing from about 1×10³ to about 1×10¹⁴ colony forming units of the strain/kg of composition.

107. The probiotic composition according to any one of items 102 to 106 for use in the treatment and/or prevention of a microbial infection or colonization by an enteropathogenic and/or foodborne pathogenic bacterium, such as C. jejuni, in a subject.

108. The probiotic composition for use according to item 107, wherein the enteropathogenic and/or foodborne pathogenic bacterium is a gram-negative bacterium.

109. The probiotic composition for use according to item 107 or 108, wherein the enteropathogenic and/or foodborne pathogenic bacterium is a bacterium of the family Campylobacteraceae or Enterobacteriaceae.

110. The probiotic composition for use according to any one of items 107 to 109, wherein the enteropathogenic and/or foodborne pathogenic bacterium is a bacterium of the family Campylobacteraceae.

111. The probiotic composition for use according to any one of items 107 to 109, wherein the enteropathogenic and/or foodborne pathogenic bacterium is a bacterium of the genus Campylobacter.

112. The probiotic composition for use according to any one of items 107 to 109, wherein the enteropathogenic and/or foodborne pathogenic bacterium is a bacterium selected from the group consisting of: C. jejuni, C. coli, C. concisus, C. fetus, C. hyoilei, C. helveticus, C. hyointestinalis, C. lari, C. mucosalis, C. sputorum and C. upsaliensis.

113. The probiotic composition for use according to any one of items 107 to 109, wherein the enteropathogenic and/or foodborne pathogenic bacterium is a bacterium of the species C. jejuni or C. coli.

114. The probiotic composition for use according to any one of items 107 to 109, wherein the enteropathogenic and/or foodborne pathogenic bacterium is a bacterium of the species C. jejuni.

115. The probiotic composition for use according to any one of items 107 to 109, wherein the enteropathogenic and/or foodborne pathogenic bacterium is a bacterium of the family Enterobacteriaceae.

116. The probiotic composition for use according to any one of items 107 to 109, wherein the enteropathogenic and/or foodborne pathogenic bacterium is a bacterium of the genus Listeria, Escherichia, Yersinia, Vibrio, Salmonella or Shigella.

117. The probiotic composition for use according to any one of items 107 to 109, wherein the enteropathogenic and/or foodborne pathogenic bacterium is a bacterium selected from the group consisting of: Listeria monocytogenes, Yersinia enterocolitica, Vibrio parachaemoliticus, Escherichia coli, Salmonella enterica, including Salmonella Enteritidis and Salmonella Infantis, and Shigella dysenteriae.

118. Use of the B. subtilis strain PS-216, or a derivative thereof having all of the identifying characteristics of B. subtilis PS-216, as a probiotic ingredient (DFM) in a feed or food product.

119. A method of preparing a feed or food composition according to any one of items 81 to 101, comprising mixing the B. subtilis strain PS-216, or a derivative thereof having all of the identifying characteristics of B. subtilis PS-216, with at least one feed ingredient.

120. Method for inhibiting, reducing or preventing biofilm formation or buildup of an enteropathogenic and/or foodborne pathogenic bacterium, such as C. jejuni, on an abiotic surface, comprising applying the B. subtilis strain PS-216, or a derivative thereof having all of the identifying characteristics of B. subtilis PS-216, to an abiotic surface.

121. Use of B. subtilis strain PS-216, or a derivative thereof having all of the identifying characteristics of B. subtilis PS-216, for inhibiting, reducing or preventing biofilm formation or buildup of an enteropathogenic and/or foodborne pathogenic bacterium, such as C. jejuni, on an abiotic surface.

124. The method according to item 120 or the use according to item 121, wherein the enteropathogenic and/or foodborne pathogenic bacterium is a gram-negative bacterium.

125. The method according to item 120 or the use according to item 121, wherein the enteropathogenic and/or foodborne pathogenic bacterium is a bacterium of the family Campylobacteraceae or Enterobacteriaceae.

126. The method according to item 120 or the use according to item 121, wherein the enteropathogenic and/or foodborne pathogenic bacterium is a bacterium of the family Campylobacteraceae.

127. The method according to item 120 or the use according to item 121, wherein the enteropathogenic and/or foodborne pathogenic bacterium is a bacterium of the genus Campylobacter.

128. The method according to item 120 or the use according to item 121, wherein the enteropathogenic and/or foodborne pathogenic bacterium is a bacterium selected from the group consisting of: C. jejuni, C. coli, C. concisus, C. fetus, C. hyoilei, C. helveticus, C. hyointestinalis, C. lari, C. mucosalis, C. sputorum and C. upsaliensis.

129. The method according to item 120 or the use according to item 121, wherein the enteropathogenic and/or foodborne pathogenic bacterium is a bacterium of the species C. jejuni or C. coli.

130. The method according to item 120 or the use according to item 121, wherein the enteropathogenic and/or foodborne pathogenic bacterium is a bacterium of the species C. jejuni.

131. The method according to item 120 or the use according to item 121, wherein the enteropathogenic and/or foodborne pathogenic bacterium is a bacterium of the family Enterobacteriaceae.

132. The method according to item 120 or the use according to item 121, wherein the enteropathogenic and/or foodborne pathogenic bacterium is a bacterium of the genus Listeria, Escherichia, Yersinia, Vibrio, Salmonella or Shigella.

133. The method according to item 120 or the use according to item 121, wherein the enteropathogenic bacterium is a bacterium selected from the group consisting of: Listeria monocytogenes, Yersinia enterocolitica, Vibrio parachaemoliticus, Escherichia coli, Salmonella enterica, including Salmonella Enteritidis and Salmonella Infantis, and Shigella dysenteriae.

134. The method or use according to any one of items 120 to 133, wherein the abiotic surface is a surface made of a material selected from the group consisting of stainless steel, tin, aluminum, titanium, chromium, plastic, glass, silicate, ceramics, and combinations thereof.

135. The method or use according to any one of items 120 to 133, wherein the abiotic surface is a water surface.

### BRIEF DESCRIPTION OF THE DRAWINGS

FIG. 1: Concentration of A) C. jejuni NCTC 11168 in mono-culture (empty bar, Control) and co-cultivated with 15 B. subtilis strains (full bars), and B) 15 C. jejuni strains from slaughterhouse environment (S1-5), human feces (H1-5) and water (W1-5), in mono-culture (empty bars) and co-cultivated with B. subtilis PS-216 (full bars), after 24 h cultivation in MH broth at 42° C. in microaerophilic conditions. Presented are average log₁₀ CFU/mL values of three replicas±standard deviation. *p<0.05.

FIG. 2: Schematic representation of broiler colonization with C. jejuni 11168 and B. subtilis PS-216 treatment experiment.

FIG. 3: Number of Bacillus spores in broiler feces before (Day 0), and 5, 8 and 11 days after inoculation with C. jejuni 11168, in not treated (hollow bars), pre-treated (horizontal line bars), continuously treated (full bars) and post-treated (vertical line bar) broilers. Average log₁₀ CFU/mL of all broilers of one group with standard deviation is presented. *p<0.05.

FIG. 4: Numbers of C. jejuni (A) and Bacillus spores (B) in broiler chickens cecum content at 21 days of age from non-treated broilers (No-treat.; circles), broilers treated with B. subtilis PS-216 spore suspension before (Pre-treatment; squares), continuously (Continuous treatment; triangles facing up) and after (Post-treatment; triangles facing down) inoculation with C. jejuni 11168. Log₁₀ CFU/g of cecum content is presented by one point per broiler. Average of one group is presented by long horizontal line, and standard deviations with shorter horizontal lines. *p<0.01.

FIG. 5: Weights of broiler chickens in kg, at 21 days of age from non-treated broilers (No-treatment), broilers treated with B. subtilis PS-216 spore suspension before (Pre-treatment), continuously (Continuous treatment) and after (Post-treatment) inoculation with C. jejuni 11168. *p<0.05.

FIG. 6: Reduction (%) of pathogenic bacteria L. monocytogenes, S. Enteritidis, S. Infantis and E. coli by B. subtilis PS-216 and B. subtilis T16-10, presented as average reduction with standard deviation, in co-culture, compared to mono-culture, after 24 h incubation at 42° C. in microaerobic conditions.

FIG. 7: Experimental design and B. subtilis-C. jejuni spatial distribution in biofilm and broth suspension during 24 h cultivation. (A) Growth rate of C. jejuni measured as CFU/mL during static mono- and coculture. Samples containing biofilm and broth were vortexed prior to plating at indicated time points (disruptive sampling). (B) right of experimental scheme, graph present differ in adhesiveness of C. jejuni to abiotic surface during co-culture with B. subtilis PS-216 compared to C. jejuni monoculture measured as CFU/mL. (C) Examined by confocal microscopy, C. jejuni in monoculture forms a submerged biofilm (green cells, left) and B. subtilis PS-216 monoculture forms a submerged biofilm on the well bottom and a pellicle at the air-liquid interface (right). PS-216 dominates in mixed culture (middle) excluding C. jejuni submerged biofilm. On right, below experimental scheme, graph presents spatial distribution of C. jejuni measured as CFU/mL during static mono- and coculture. (D) Growth rate of C. jejuni measured as CFU/mL during static mono- and coculture by growing pairs of strains on either side of a 0.1 μm permeable membrane. Three biological and up to three technical replicas were used. Error bars are displayed as mean values±the standard deviation of the mean value. * represent statistically significant values (Two-Sample t-test).

FIG. 8: Experimental scheme and B. subtilis-C. jejuni spatial distribution in biofilm and broth suspension during 24 h cultivation. (A) Growth rate of B. subtilis measured as CFU/mL during static mono- and coculture. Samples containing biofilm and broth were vortexed prior to plating at indicated time points (disruptive sampling). (B) right of experimental scheme, graph present differ in adhesiveness of B. subtilis to abiotic surface during co-culture with C. jejuni compared to B. subtilis monoculture measured as CFU/mL. (C) Below experimental scheme, graph presents spatial distribution of PS-216 measured as CFU/mL during static mono- and coculture. (D) Growth rate of B. subtilis measured as CFU/mL during static mono- and coculture by growing pairs of strains on either side of a 0.1 μm permeable membrane. Three biological and up to three technical replicas were used. Error bars are displayed as mean values±the standard deviation of the mean value. * represent statistically significant values (Two-Sample t-test).

FIG. 9: B. subtilis PS-216 overrides the pre-established submerged C. jejuni biofilm. On the top right corner is presented experimental scheme of confocal experiments. Growth rate of C. jejuni measured as CFU/mL during static mono- and coculture biofilm assay. B. subtilis was introduced (t₀) to undisturbed C. jejuni biofilms pre-cultivated for 26 h and C. jejuni ratio advantage was 1:10000. Samples containing biofilm and broth were vortexed prior to plating at indicated time points. Co-culture was incubated and sampled at 26 h (t₀—start of treatment with PS-216), 38 h/(t₁₂) and 48 h of co-incubation. Part of the graph below the CFU values, represent pre-established submerged C. jejuni monoculture biofilm using brightfield and confocal micrososcopy (A-green cells) before treatment (to), and in co-culture with B. subtilis after 12 h treatment (t₁₂) (C—red cells) compared to C. jejuni monoculture biofilm (B—green cells). Visualization of the B. subtilis effect on C. jejuni pre-established biofilm revealed strong PS-216 effect excluding C. jejuni from the characteristic niche position. Three biological and up to three technical replicas were used. Error bars are displayed as mean values±the standard deviation of the mean value. * represent statistically significant values (Two-Sample t-test).

FIG. 10: (A) C. jejuni showed no effect to B. subtilis PS-216 during biofilm assay. Growth rate of B. subtilis measured as CFU/mL during static mono- and coculture biofilm assay. Samples containing biofilm and broth were vortexed prior to plating at indicated time points. (B) B. subtilis PS-216 is able to disintegrate the pre-established C. jejuni biofilm in as little as 12 h of co-incubation in microaerobic conditions, leaving no visible aggregates and finger-like structures (1. and 2. TRETAED), characteristic for C. jejuni submerged biofilms. Three biological and up to three technical replicas were used. Error bars are displayed as mean values±the standard deviation of the mean value. * represent statistically significant values (Two-Sample t-test).

### DETAILED DESCRIPTION OF THE INVENTION

Unless specifically defined herein, all technical and scientific terms used have the same meaning as commonly understood by a skilled person.

All methods and materials similar or equivalent to those described herein can be used in the practice or testing of the present invention, with suitable methods and materials being described herein. All publications, patent applications, patents, and other references mentioned herein are incorporated by reference in their entirety. In case of conflict, the present specification, including definitions, will prevail. Further, the materials, methods, and examples are illustrative only and are not intended to be limiting, unless otherwise specified.

The present invention generally related to the Bacillus subtilis strain PS-216, which was isolated from the riverbank soil of the river Sava in Slovenia and further characterized by Štefanič and Mandić-Mulec (2009). The Bacillus subtilis strain PS-216 has been deposited with National Collection of Agricultural and Industrial Microorganisms (NCAIM), Budapest, Hungary, on May 22, 2020, under the provisions of the Budapest Treaty on the International Recognition of the Deposit of Microorganisms for the Purpose of Patent Procedure under accession no. B 001478.

As noted above, the present invention is based inter alia on the surprising finding that the Bacillus subtilis strain PS-216 shows strong inhibitory activity against Campylobacter jejuni. Particularly, the present inventors have found that B. subtilis PS-216 greatly reduce C. jejuni colonization in broilers compared to other B. subtilis strains. Moreover, it has been shown that the treatment of broilers with B. subtilis PS-216 results in an increased weight of broilers.

Based on these surprising properties, B. subtilis PS-216 is an excellent strain to be used as a probiotic, especially in animal husbandry, since it improves the health status and other physical parameters. The function of a probiotics (also called “direct-fed microbials” or “DFM”) is to influence the gut microflora in a positive way by supporting the growth of beneficial bacteria and/or the suppression of the growth of pathogenic bacteria, such as C. jejuni. Ideally, by using probiotics the use of antibiotic growth promotors (AGPs) becomes redundant. Both aspects are met by B. subtilis PS-216.

The present invention thus provides the use of the B. subtilis strain PS-216, or a derivative thereof having all of the identifying characteristics of B. subtilis PS-216, in inhibiting, reducing or preventing the colonization of an enteropathogenic and/or foodborne pathogenic bacterium, such as C. jejuni, in a subject.

The present invention further provides a method for inhibiting, reducing or preventing the colonization of an enteropathogenic and/or foodborne pathogenic bacterium, such as C. jejuni, in a subject, comprising administering an effective amount of the B. subtilis strain PS-216, or a derivative thereof having all of the identifying characteristics of B. subtilis PS-216, to the subject.

According to certain embodiments, the bacterium being the target of the present invention is an enteropathogenic bacterium. The enteropathogenic bacterium being the target of the present invention may be any bacterium tending to produce a disease in the intestinal tract of a subject. According to certain embodiments, the bacterium being the target of the present invention is a foodborne pathogenic bacterium. The foodborne pathogenic bacterium being the target of the present invention may be any bacterium causing foodborne illness, either directly or indirectly via a toxic substance produced by it. According to certain embodiments, the enteropathogenic bacterium is also foodborne pathogenic.

The enteropathogenic and/or foodborne pathogenic bacterium may be gram-positive or gram-negative.

According to certain embodiments, the enteropathogenic and/or foodborne pathogenic bacterium is a gram-negative bacterium.

According to certain embodiments, the enteropathogenic and/or foodborne pathogenic bacterium is a bacterium of the family Campylobacteraceae or Enterobacteriaceae.

According to certain embodiments, the enteropathogenic and/or foodborne pathogenic bacterium is a bacterium of the family Campylobacteraceae.

According to certain embodiments, the enteropathogenic and/or foodborne pathogenic bacterium is a bacterium of the genus Campylobacter.

According to certain embodiments, the enteropathogenic and/or foodborne pathogenic bacterium is a bacterium selected from the group consisting of: C. jejuni, C. coli, C. concisus, C. fetus, C. hyoilei, C. helveticus, C. hyointestinalis, C. lari, C. mucosalis, C. sputorum and C. upsaliensis.

According to certain embodiments, the enteropathogenic and/or foodborne pathogenic bacterium is a bacterium of the species C. jejuni or C. coli.

According to certain embodiments, the enteropathogenic and/or foodborne pathogenic bacterium is a bacterium of the species C. jejuni.

According to certain embodiments, the enteropathogenic and/or foodborne pathogenic bacterium is a bacterium of the family Enterobacteriaceae.

According to certain embodiments, the enteropathogenic bacterium is a bacterium of the genus Listeria, Escherichia, Yersinia, Vibrio, Salmonella or Shigella.

According to certain embodiments, the enteropathogenic bacterium is a bacterium selected from the group consisting of: Listeria monocytogenes, Yersinia enterocolitica, Vibrio parachaemoliticus, Escherichia coli, Salmonella enterica, including Salmonella Enteritidis and Salmonella Infantis, and Shigella dysenteriae.

The present invention further provides the use of the B. subtilis strain PS-216, or a derivative thereof having all of the identifying characteristics of B. subtilis PS-216, in improving the health status, such as the gut health status, of a subject.

The present invention further provides a method for improving the health status, such as the gut health status, of a subject, comprising administering an effective amount of the B. subtilis strain PS-216, or a derivative thereof having all of the identifying characteristics of B. subtilis PS-216, to the subject.

The present invention further provides the use of the B. subtilis strain PS-216, or a derivative thereof having all of the identifying characteristics of B. subtilis PS-216, in enhancing the growth of a non-human animal.

The present invention further provides a method for enhancing the growth of a non-human animal, such as poultry animal, comprising administering an effective amount of the B. subtilis strain PS-216, or a derivative thereof having all of the identifying characteristics of B. subtilis PS-216, to the non-human animal.

The present invention further provides the use of in increasing the weight of a non-human animal.

The present invention further provides a method for increasing the weight of a non-human animal, such as poultry animal, comprising administering an effective amount of the B. subtilis strain PS-216, or a derivative thereof having all of the identifying characteristics of B. subtilis PS-216, to the non-human animal.

The methods and uses of the strain can be therapeutic or non-therapeutic. According to particular embodiments, the methods and uses are non-therapeutic, in particular feeding applications.

The subject that may benefit from the present invention may be a human or non-human animal. According to certain embodiments, the subject is a human. According to certain embodiments, the subject is a non-human animal.

The non-human animal that may benefit from the present invention include, but are not limited to farm animals, pets, exotic animals and zoo animals. The non-human animal may thus be a farm animal, which is raised for consumption or as food-producers, such as poultry, swine and ruminants.

According to certain embodiments, the non-human animal is productive animal. The productive animal may be selected from the group consisting of pig, chicken, duck, goose, turkey, cow, sheep and goat.

According to certain embodiments, the non-human animal is a poultry animal.

The poultry animal may be selected from productive or domestic poultry, but also from fancy poultry or wild fowl. Productive poultry animals of particular interested are chickens, turkeys, ducks and geese. Fancy poultry or wild fowl are peacocks, pheasants, partridges, chukkars, guinea fowl, quails, capercaillies, grouse, pigeons and swans.

According to certain embodiments, the poultry animal is a productive poultry animal.

According to some embodiments, the poultry animal is selected from the group consisting of: chicken, duck, goose, and turkey, guinea fowl and pigeon.

According to some embodiments, the poultry animal is a chicken.

The non-human animal being the subject of the present invention may be of any age, but suitable is of young age. Thus, according to certain embodiments, the non-human animal is under four weeks old.

According to some embodiments, the non-human animal is between 1 and 21 days old.

According to some embodiments, the non-human animal is between 1 and 14 days old.

According to some embodiments, the non-human animal is between 1 and 7 days old.

According to some embodiments, the non-human animal is between 13 and 21 days old.

The strain B. subtilis strain PS-216, or a derivative thereof having all of the identifying characteristics of B. subtilis PS-216, may be administered or used in any suitable form, such as in the form of spores, cells, vegetative cells or a dried cell mass.

However, spores are most suitable since they generally show higher resistance to gastric conditions and bile salt compared to other forms, such as vegetative cells, and thus have an overall higher survival rate (as demonstrated in Example 1). Thus, according to certain embodiments, the strain is administered or used in the form of spores.

The strain B. subtilis strain PS-216, or a derivative thereof having all of the identifying characteristics of B. subtilis PS-216, may be administered or used in any effective amount. Suitably, the strain is administered or used at a bacterial load of at least about 1×10² colony forming units (CFU). According to certain embodiments, the strain is administered or used at a bacterial load in a range from about 1×10² to about 1×10¹⁴ colony forming units. According to certain embodiments, the strain is administered or used at a bacterial load in a range from about 1×10⁴ to about 1×10¹⁰ colony forming units. According to certain embodiments, the strain is administered or used at a bacterial load in a range from about 1×10⁴ to about 1×10⁹ colony forming units. According to certain embodiments, the strain is administered or used at a bacterial in a range from about 1×10⁴ to about 1×10⁸ colony forming units.

The B. subtilis strain PS-216, or a derivative thereof having all of the identifying characteristics of B. subtilis PS-216, may be administered or used in liquid form, for example by spraying, or as a powder, for example by strewing. It may also be administered or used as part of a feed composition or as drinking or rearing water. Thus, the strain may be administered or used perorally. It may however also be administered or used by spraying or by aerosol.

According to certain embodiments, the strain is administered or used in an aqueous solution, such as drinking water. According to some embodiments, the strain is administered or used in a water solution containing from about 1×10⁴ to about 1×10⁸ CFU/mL, such as about 2.5×10⁶ CFU/mL, B. subtilis PS-216 spores as drinking water.

According to some embodiment, the strain is administered or used as part of a feed composition, such as a feed composition of the present invention.

The present invention thus further provides a feed or food composition containing the B. subtilis strain PS-216, or a derivative thereof having all of the identifying characteristics of B. subtilis PS-216, and at least one further feed or food ingredient.

Typical feed or food ingredients which may be contained in the compositions according to the invention and/or used in the preparation of such feed or food compositions include one or more of the following (but are not limited thereto): proteins, carbohydrates, fats, fibers, further probiotics, prebiotics, enzymes, vitamins, minerals, amino acids, and combinations thereof.

Thus, according to certain embodiments, the feed or food ingredient is selected from the group consisting of proteins, carbohydrates, fats, fibers, further probiotics, prebiotics, enzymes, vitamins, minerals, amino acids, and combinations thereof.

Carbohydrates useful in the context of the invention may, for example, be forage, roughage, wheat meal, sunflower meal or soya meal, and mixtures thereof. Proteins useful in the context of the invention may, for example, be soya protein, pea protein, wheat gluten or corn gluten, and mixtures thereof.

Fats useful in the context of the invention may, for example, be particular oils, of both animal and plant origin, like vegetable oils, for example soya bean oil, rapeseed oil, sunflower seed oil, flaxseed oil or palm oil, fish oil, and mixtures thereof. Proteins which additionally contain fats which may be also be useful are for example fish meal, krill meal, bivalve meal, squid meal or shrimp shells, as well as combinations thereof.

Fibers useful in the context of the invention may, for example, be non-starch polysaccharides and other plant components such as cellulose, resistant starch, resistant dextrins, inulin, lignins, chitins, pectins, beta-glucans, and oligosaccharides.

Further probiotics (DFM) useful in the context of the invention may, for example, be other bacteria, such as those selected from the species Bacillus subtilis, Lactobacillus spp., Bifidobacterium spp., Enterococcus spp., Streptococcus spp., Pediococcus spp., Leuconostoc spp., Saccharomyces spp.

Prebiotics useful in the context of the invention may, for example, be oligosaccharides, in particular selected from galactooligosaccharides, silayloligosaccharides, lactulose, lactosucrose, fructooligosaccharides, palatinose or isomaltose oligosaccharides, glycosyl sucrose, maltooligosaccharides, isomaltooligosaccharides, cyclodextrins, gentiooligosaccharides, soybean oligosaccharides, xylooligosaccharides, dextrans, pectins, polygalacturonan, rhamnogalacturonan, mannan, hemicellulose, arabinogalactan, arabinan, arabinoxylan, resistant starch, mehbiose, chitosan, agarose, inulin, tagatose, polydextrose, or alginate.

Enzymes useful in the context of the invention may, for example, be phytases (EC 3.1.3.8 or 3.1.3.26), xylanases (EC 3.2.1.8), galactanases (EC 3.2.1.89), galactosidases, in particular alpha-galactosidases (EC 3.2.1.22), proteases (EC 3.4), phospholipases, in particular phospholipases Al (EC 3.1.1.32), A2 (EC 3.1.1.4), C (EC 3.1.4.3), and D (EC 3.1.4.4), lysophospholipases (EC 3.1.1.5), amylases, in particular alpha-amylases (EC 3.2.1.1); lysozymes (EC 3.2.1.17), glucanases, in particular beta-glucanases (EC 3.2.1.4 or EC 3.2.1.6

Examples of commercially available phytases include Bio-Feed™ Phytase (Novozymes), Ronozyme® P and HiPhos™ (DSM Nutritional Products), Natuphos™ (BASF), Finase® and Quantum® Blue (AB Enzymes), the Phyzyme® XP (Verenium/DuPont) and Axtra® PHY (DuPont).

Vitamins useful in the context of the invention may, for example, be vitamin A, vitamin D3, vitamin E, vitamin K, e.g., vitamin K3, vitamin B 12, biotin, choline, vitamin BI, vitamin B2, vitamin B6, niacin, folic acid and panthothenate, e.g., Ca-D-panthothenate, or combinations thereof.

Minerals useful in the context of the invention may, for example, be for example boron, cobalt, chloride, chromium, copper, fluoride, iodine, iron, manganese, molybdenum, selenium, zinc, calcium, magnesium, potassium, or sodium, or combinations thereof.

Amino acids useful in the context of the invention may, for example, be any naturally occurring amino acids, and more specifically, any of the essential amino acids histidine, isoleucine, lysine, methionine, phenylalanine, threonine, tryptophan, valine or combinations thereof.

According to some embodiments, the feed or food composition of the present invention comprises at least one vitamin, such as Vitamin A, Vitamin E or a combination thereof.

According to some embodiments, the feed or food composition of the present invention comprises at least one mineral, such as calcium, phosphorus, manganese, sodium (such as in the form of sodium chloride) or any combination thereof.

According to some embodiments, the feed or food composition of the present invention comprises at least one amino acid, such as at least one naturally occurring amino acid.

According to some embodiments, the feed or food composition of the present invention comprises at least one essential amino acid, such as lysine, methionine or a combination thereof.

According to some embodiments, the feed or food composition of the present invention comprises at least one fat, such as crude fat.

According to some embodiments, the feed or food composition of the present invention comprises at least one fiber, such as crude fiber.

According to some embodiments, the feed or food composition of the present invention comprises at least one protein, such as crude protein.

According to some embodiments, the feed or food composition of the present invention comprises at least one enzyme, such as a phytase.

The strain B. subtilis strain PS-216, or a derivative thereof having all of the identifying characteristics of B. subtilis PS-216, may be contained in the feed or food composition in any suitable form, such as in the form of spores, cells, vegetative cells or a dried cell mass.

However, as noted above, spores are most suitable since they generally show higher resistance to gastric conditions and bile salt compared to other forms, such as vegetative cells, and thus have an overall higher survival rate (as demonstrated in Example 1). Thus, according to certain embodiments, the strain is in the form of spores.

Suitably, the strain is present at a bacterial load of at least about 1×10² colony forming units (CFU) per kg of composition. According to some embodiments, the feed or food composition contains at least about 1×10³ colony forming units (CFU) of the strain/kg of composition. According to certain embodiments, the feed or food composition contains from about 1×10³ to about 1×10¹⁴ colony forming units of the strain/kg of composition. According to certain embodiments, the feed or food composition contains from about 1×10³ to about 1×10¹⁰ colony forming units of the strain/kg of composition. According to certain embodiments, the feed or food composition contains from about 1×10³ to about 1×10⁹ colony forming units of the strain/kg of composition. According to certain embodiments, the feed or food composition contains from about 1×10³ to about 1×10⁸ colony forming units of the strain/kg of composition.

According to certain embodiments, the strain is comprised in an amount of 0.01 to 10 wt.-%, such as in an amount of 0.02 to 5 wt.-%, in particular in an amount of 0.03 to 3 wt.-%. According to certain embodiments, the strain is comprised in an amount of 0.05 to 3 wt.-%. According to certain embodiments, the strain is comprised in an amount of 0.1 to 3 wt.-%. According to certain embodiments, the strain is comprised in an amount of 0.5 to 3 wt.-%. According to certain embodiments, the strain is comprised in an amount of 1 to 3 wt.-%.

The present invention further provides a method of preparing a feed or food composition according to the invention, comprising mixing the B. subtilis strain PS-216, or a derivative thereof having all of the identifying characteristics of B. subtilis PS-216, with at least one feed ingredient.

The present invention further provides a probiotic composition comprising the B. subtilis strain PS-216, or a derivative thereof having all of the identifying characteristics of B. subtilis PS-216, and at least one excipient, such as at least one pharmaceutically or agriculturally acceptable excipient.

Like with the feed or food composition, the strain may be contained in probiotic composition in any suitable form, such as in the form of spores, cells, vegetative cells or a dried cell mass, but most suitable is in the form of spores.

Suitably, the strain is present at a concentration of at least about 1×10² colony forming units (CFU) per kg of composition. According to some embodiments, the probiotic composition contains at least about 1×10³ colony forming units (CFU) of the strain/kg of composition. According to certain embodiments, the probiotic composition contains from about 1×10³ to about 1×10¹⁴ colony forming units of the strain/kg of composition. According to certain embodiments, the probiotic composition contains from about 1×10³ to about 1×10¹⁰ colony forming units of the strain/kg of composition. According to certain embodiments, the probiotic composition contains from about 1×10³ to about 1×10⁹ colony forming units of the strain/kg of composition. According to certain embodiments, the probiotic composition contains from about 1×10³ to about 1×10⁸ colony forming units of the strain/kg of composition.

According to certain embodiments, the strain is comprised in an amount of 0.01 to 10 wt.-%, such as in an amount of 0.02 to 5 wt.-%, in particular in an amount of 0.03 to 3 wt.-%. According to certain embodiments, the strain is comprised in an amount of 0.05 to 3 wt.-%. According to certain embodiments, the strain is comprised in an amount of 0.1 to 3 wt.-%. According to certain embodiments, the strain is comprised in an amount of 0.5 to 3 wt.-%. According to certain embodiments, the strain is comprised in an amount of 1 to 3 wt.-%.

Because of the strong inhibitory activity against C. jejuni shown by Bacillus subtilis PS-216, the probiotic composition of the present invention is particularly useful in the treatment and/or prevention of a microbial infection or colonization by C. jejuni in a human or non-human animal, such as a poultry animal.

Further, B. subtilis PS-216, or a derivative thereof having all of the identifying characteristics of B. subtilis PS-216, may be used as a probiotic ingredient (DFM) in a feed or food product.

The present inventors have further found that B. subtilis PS-216 inhibits the formation of biofilms of an enteropathogenic and/or foodborne pathogenic bacterium, such as C. jejuni, on abiotic surfaces and is able to disintegrate preestablished biofilm. Biofilm is a surface attached form of bacterial growth that is responsible for a large number of life-threatening diseases.

Thus, the present invention provides the use of B. subtilis strain PS-216, or a derivative thereof having all of the identifying characteristics of B. subtilis PS-216, for inhibiting, reducing or preventing biofilm formation or buildup of an enteropathogenic and/or foodborne pathogenic bacterium on an abiotic surface.

The present invention further provides a method for inhibiting, reducing or preventing biofilm formation or buildup of an enteropathogenic and/or foodborne pathogenic bacterium on a surface, comprising applying the B. subtilis strain PS-216, or a derivative thereof having all of the identifying characteristics of B. subtilis PS-216, to an abiotic surface.

The enteropathogenic and/or foodborne pathogenic bacterium may be one as defined above.

The abiotic surface may be any surface made of stainless steel, tin, aluminum, titanium, chromium, plastic, glass, silicate, ceramics, or any combination thereof.

According to certain embodiments, the abiotic surface is made of stainless steel.

According to certain embodiments, the abiotic surface is made of plastic. The plastic may be made of polyvinylchlorise or polystyrene.

According to certain embodiments, the abiotic surface is a water surface.

The strain B. subtilis strain PS-216, or a derivative thereof having all of the identifying characteristics of B. subtilis PS-216, may be applied in any suitable form, such as in the form of spores, cells or vegetative cells.

The B. subtilis strain PS-216, or a derivative thereof having all of the identifying characteristics of B. subtilis PS-216, may be applied in liquid form. According to certain embodiment, the B. subtilis strain PS-216, or a derivative thereof having all of the identifying characteristics of B. subtilis PS-216, is applied in an aqueous solution.

The B. subtilis strain PS-216, or a derivative thereof having all of the identifying characteristics of B. subtilis PS-216, may be applied by any suitable technique, such as by spraying, by aerosol, by pouring, or by means of a brush.

Having generally described this invention, a further understanding can be obtained by reference to certain specific examples, which are provided herein for purposes of illustration only, and are not intended to be limiting unless otherwise specified.

### EXAMPLES

### Example 1

In an effort to find the most suitable B. subtilis strain against C. jejuni in co-culture, we have tested 15 B. subtilis strains and chosen one, B. subtilis PS-216, with the best anti-Campylobacter activity. This strain we evaluated further against 15 C. jejuni strains, determined its antimicrobial resistance profile and its ability to survive in the gut environment with simulated intestinal conditions. Finally, we evaluated the ability of B. subtilis PS-216 to prevent or reduce C. jejuni colonization of broilers by addition of B. subtilis PS-216 into the broilers water supply and determined the influence of spore treatment on broiler weight.

Material and Methods

Strains and Growth Conditions

C. jejuni isolated from human feces, surface water and slaughter house environment, and characterized by Kovač et al. (2018), C. jejuni 11168 (Kovač et al., 2015), and B. subtilis strains isolated from the riverbank soil of the river Sava in Slovenia and characterized by Štefanič and Mandič-Mulec (2009), were stored at −80° C. in a solution of 20% glycerol and 80% Mueller Hinton broth (HMB; Oxoid, UK). C. jejuni strains were revitalized and grown on Mueller Hinton agar (MHA; Oxoid, UK) at 42° C. in microaerophilic conditions (5% O2, 10% C02, and 85% N2) for 24 hours. B. subtilis strains were grown on MHA or in LB medium (BD Difco, USA) at 37° C. in aerobic conditions. The second passage was used in experiments. To enumerate C. jejuni in mono- and co-cultures, Karmali agar (Oxoid, UK) supplemented with Karmali selective supplement (SR0167E; Oxoid, UK) was used. MHA was supplemented with Bolton broth selective supplement (SR0183; Oxoid, UK) and growth supplement (SR0232E; Oxoid, UK) (MHA-BSS), when appropriate. For enumeration of Bacillus sp. from fecal samples HiChrom Bacillus agar (HCBA; Himedia, USA) was used.

Co-Cultivation of B. subtilis with C. jejuni in Mueller Hinton Broth

B. subtilis strains (n=15) were cultivated together with C. jejuni 11168 in 1:50 starting ratio in favor of C. jejuni in MH broth. Co-cultures and control monocultures were cultivated at 42° C. in microaerobic conditions for 24 hours. B. subtilis strain PS-216 was chosen for further experiments and its anti-Campylobacter effect was tested on 15 C. jejuni strains from slaughterhouse environment (S1-5; n=5), human feces (H1-5; n=5), and surface water environment (W1-5; n=5), in co-culture (42° C., microaerobic conditions, 24 h). Co-cultivation experiments were carried out in three biological and up to three technical replicas.

Bacillus subtilis Spore Preparation

B. subtilis spores were prepared according to Warriner and Waites (1999) with some modifications. Briefly, bacteria were grown in Luria-Bertani (LB) (CONDA, Spain) medium (200 rpm, 37° C.) over night and further diluted 100-times and incubated four days in sporulation media, which contained 16 g/L Nutrient broth (Oxoid, UK), 2 g/L KCl (Fisher Scientific, USA), 1 mM MgSO4 (Oxoid, UK), 1 mM CaCl2 (Merck, Germany), 1 μM FeSO4 (Sigma Aldrich, Germany), 10 μM MnCl2 (Sigma Aldrich, Germany), and 2.8 mM D-(+)-glucose (Sigma Aldrich, Germany). The culture was treated at 80° C. for 30 min and washed with 0.9% NaCl (10000 g, 10 min) three times and stored with 10% glycerol at −20° C. until further use.

Determination of the Susceptibility of the Studied B. subtilis to Antimicrobials: Determination of Minimal Inhibitory Concentrations (MIC)

For 8 antimicrobials of human or veterinary importance (tetracycline, chloramphenicol, kanamycin, erythromycin, streptomycin, gentamycin, tylosin tartrate and ampicillin; Sigma Aldrich, Germany), minimal inhibitory concentrations (MICs) were determined by microwell dilution method using MH broth. Two-fold serial dilutions of antibiotics were prepared in concentrations ranging between 0.5-64 mg/ml. The assays were performed in 96 well plates; in each well, 50 μL of appropriate dilutions of antibiotics were added to 50 μL of bacterial suspension previously diluted in MH broth to 105 CFU/mL. The MICs were expressed as the lowest concentration of the antibiotic with no visible growth of bacteria. Breakpoint values for antimicrobials for Bacillus spp. were used as described by the European Food Safety Agency (EFSA, 2012).

Acid and Bile Salt Tolerance of Vegetative Cells and Spores

Resistance of vegetative cells and spores to simulated gastric conditions and bile salt was determined as described by Barbosa et al. (2005) and by Mingmongkolchai and Panbangred (2017), with some modifications. Briefly, the overnight culture (16 h) in LB medium containing 1% glucose was sub-cultured (1% inoculum) into LB medium containing 1% glucose and grown at 37° C. with shaking (110 rpm) for 3 h to prevent sporulation. Vegetative cells were resuspended in 5 ml of LB acidified to pH 2.5 (with 1M HCl) and supplemented with pepsin from porcine gastric mucosa (Sigma-Aldric, Switzerland) at 1 mg/mL or LB supplemented with 0.3% (w/v) bile salts (Oxoid, UK). For spore assay, spores were resuspended in 5 ml of a 0.85% NaCl solution adjusted to pH 2.5, supplemented with pepsin at 1 mg/mL or an isotonic buffer (Oxoid, UK) supplemented with 0.3% bile salts (Oxoid, UK). Cultures were incubated at 37° C. with agitation (110 rpm) and aliquots were removed after 30, 60 and 90 min for acid tolerance, and after 60 and 180 min for bile salt tolerance. Bacterial cell survival was calculated as follows: NA/NB×100, where NA=log 10 CFU/mL after incubation and NB=log 10 CFU/mL before incubation.

Broiler Colonization Experiment

One day old broiler chicks (n=45) obtained from a commercial hatchery were divided into four groups of 11 and 12 chicks per group. Broilers were kept in tubs with soft bedding, and water and feed provided ad libitum. At the age of day 8 all broilers were inoculated with 3.94×10⁶ CFU of C. jejuni 11168 by oral gavage. Treatment was administered in water solution containing approx. 2.5×10⁶ CFU/mL B. subtilis PS-216 spores as drinking water, at appropriate times.

To evaluate the ability of B. subtilis PS-216 to prevent and/or reduce C. jejuni colonization, broilers were administered following B. subtilis (BS) treatment regimens: i) No treatment control (n=12) was inoculated with C. jejuni, but not treated with B. subtilis spores, ii) Pre-treatment group (n=11) was administered spore treatment solution before inoculation with C. jejuni, from day of age 1 to 7, iii) Continuous treatment (n=11) group received spore treatment solution throughout the experiment, from day of age 1 to 20, and iv) Post-treatment group (n=11) was administered spore treatment solution 5 days after inoculation with C. jejuni, from day of age 13 to 20.

Cloacal swabs were collected from each broiler prior to C. jejuni inoculation (Day 0) to confirm the absence of C. jejuni, five days after inoculation (Day 5) to confirm colonization with C. jejuni, and 8 and 11 days after inoculation. At 21 days of age all broiler chickens were sacrificed, weighted, and cecum content was collected. All collected swabs and cecum contents were 10 timed diluted in MH broth and plated onto MH-BSS to enumerate C. jejuni in feces and treated at 80° C. for 30 min and plated onto HCBA to enumerate Bacillus spores.

Statistical Analysis

One-way ANOVA with Tukey's post-hoc test was used to analyze the influence of B. subtilis multiple strains on one C. jejuni strain. To evaluate the influence of co-cultivation on B. subtilis growth Student's t-test (paired) was used. Two-way ANOVA with Bonferroni post hoc test was used to analyze the influence of one B. subtilis strain on multiple C. jejuni strains.

The differences between treated and untreated broilers were analyzed using Student's t-test.

Statistical analysis was performed with the SPSS software version 21 (IBM Corp., NY, USA) and GraphPad Prism software version 8 (GraphPad Software Inc., CA, USA).

Results

B. subtilis Reduces C. jejuni Growth in Co-Culture

To determine the most suitable B. subtilis strain against C. jejuni, the anti-Campylobacter effect of 15 B. subtilis strains isolated from the riverbank soil of the river Sava in Slovenia (Štefanič and Mandić-Mulec 2009) was determined against C. jejuni 11168 in liquid co-culture, with a 1:50 starting inoculum ration in favor of C. jejuni, at 42° C. in microaerobic conditions.

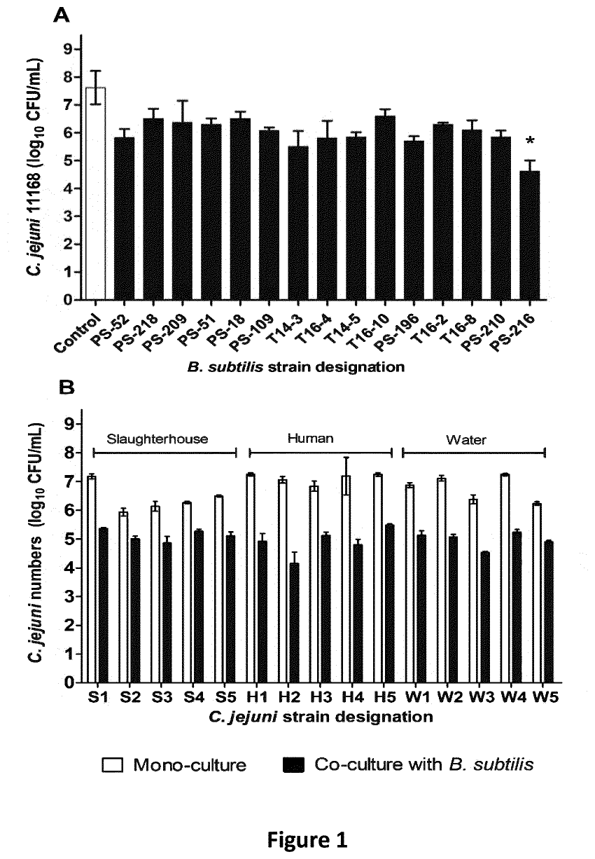

After a 24-hour cultivation we saw reduced C. jejuni growth in co-culture with B. subtilis compared to C. jejuni mono-culture, with a 1.03 to 3.01 log reduction, as seen (FIG. 1A).

The majority of B. subtilis strains decreased C. jejuni numbers significantly (p<0.05). Only strains PS-218, PS-18 and T16-10 had no significant effect on C. jejuni growth (p>0.05). B. subtilis strain PS-216 stands out amongst other strains (p<0.05) with a 3.01 log reduction of C. jejuni 11168 and was therefore chosen for further experiments.

To confirm the effect of B. subtilis PS-216 and thus its usefulness as an anti-Campylobacter agent, it was further tested in co-culture with 15 C. jejuni strains, isolated from the slaughterhouse environment (S1-5, n=5), human feces samples (H1-5, n=5), and surface water environment (W1-5, n=5). A good anti-Campylobacter effect was confirmed as significant reduction of C. jejuni growth was achieved (FIG. 1B). Log reductions ranged from 0.93 to 2.81 log for different C. jejuni strains. When comparing groups of C. jejuni strains, we found the best effect of B. subtilis PS-216 to be against human feces strains (H) with a 2.22±0.45 average log reduction, and the least effect against strains from the slaughterhouse environment (S), with a 1.28±0.32 average log reduction compared to mono-culture.

Although starting co-cultivation conditions were favorable for C. jejuni with higher staring numbers, B. subtilis managed to reduce C. jejuni growth significantly, regardless of strain origin.

B. subtilis PS-216 is Susceptible to Antimicrobials

The antimicrobial susceptibility of the studied Bacillus isolate PS-216 was tested against 8 antimicrobials with human and veterinary relevance. The effect of tetracycline, chloramphenicol, kanamycin, erythromycin, streptomycin, gentamycin, tylosin tartrate and ampicillin was tested and compared to the susceptibility of the reference strain B. subtilis ACTCC 6633 (Table 1). Both the PS-216 and the reference strain are susceptible to all tested antimicrobials. The B. subtilis PS-216 is, compared to the reference strain, less susceptible to tetracycline, streptomycin, and ampicillin with a >16-fold, 2-fold, and >4-fold difference, respectively.

TABLE 1
Susceptibility of B. subtilis PS-216 and reference
strains B. subtilis ATCC 6633 to the range of tested antibiotics,
presented as MICs and corresponding sensitivity (S) or resistance (R).

| B. subtilis | MIC of antibiotic (mg/L) and strain sensitivity (S/R)* | | | | | | | |
|---|---|---|---|---|---|---|---|---|
| isolate | TET | CHL | KN | ERY | STR | GEN | TY | AMP |
| PS-216 | 8 (S) | 2 (S) | <0.5 (S) | <0.5 (S) | 8 (S) | <0.5 (S) | <0.5 | 2 |
| ACTCC 6633 | <0.5 (S) | 2 (S) | <0.5 (S) | <0.5 (S) | 4 (S) | <0.5 (S) | <0.5 | <0.5 |

*S, sensitive; R, resistant; according to European Food Safety Agency (EFSA, 2012) for Bacillus spp. TET, tetracycline; CHL, chloramphenicol; KN, kanamycin; ERY, erythromycin; STR, streptomycin; GEN, gentamycin; TY, tylosin tartrate; AMP, ampicillin.

Given that the studied B. subtilis PS-216 are susceptible to the antimicrobials tested they can be considered as potentially safe for use as probiotic culture.

B. subtilis PS-216 is highly sensitive to simulated gastric conditions in vegetative form and highly resistant in spore form

For a probiotic to be effective against C. jejuni it has to survive the harsh gastric conditions of a subject and reach C. jejuni in the intestine. The acid and bile salt tolerance assay showed that vegetative cells of PS-216 isolate were very susceptible to simulated gastric conditions, as a 100% decrease in cell viability was observed when exposed to simulated gastric conditions at 37° C. for 30 min (Table 2). In contrast, the spores displayed 100% survival after exposure for 90 min in simulated gastric conditions and for 180 min in 0.3% bile salts. This result reveals that spores from isolate PS-216 show an excellent resistance to simulated gastric conditions and to 0.3% bile salts.

TABLE 2
Survival of B. subtilis PS-216 vegetative cells and spores in
simulated gastric conditions of pH 2.5 with 1 mg/mL pepsin and
0.3% bile salts, presented as % of cells/spores after treatment.

| | pH 2.5 + 1 mg/mL pepsin | | | | | | 0.3% bile salts | | | |
|---|---|---|---|---|---|---|---|---|---|---|
| B. subtilis | Vegetative | | | | | | Vegetative | | | |
| PS-216 | cells | | | Spores | | | cells | | Spores | |
| Treatment | 30 | 60 | 90 | 30 | 60 | 90 | 60 | 180 | 60 | 180 |
| time | | | | | | | | | | |
| (min) | | | | | | | | | | |
| Survival | 0 | 0 | 0 | 100 | 100 | 100 | 0 | 0 | 100 | 100 |
| (%) | | | | | | | | | | |

B. subtilis PS-216 Reduces C. jejuni and Alters Weight in Broilers

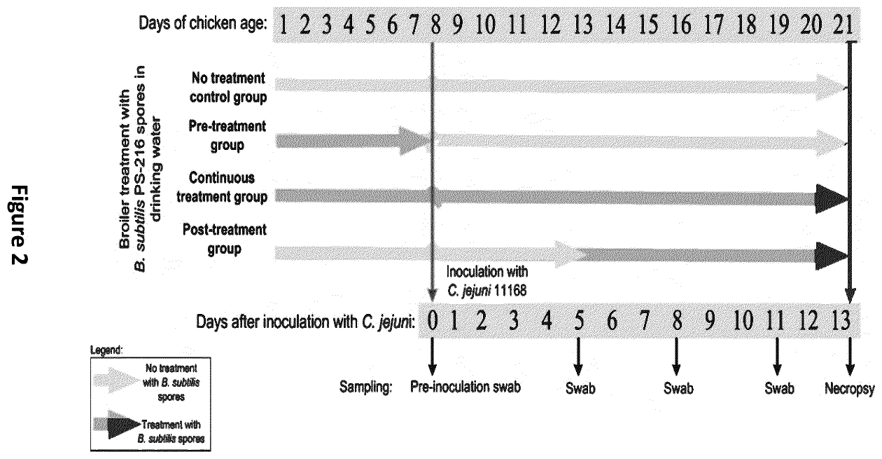

To evaluate the influence of B. subtilis PS-216 on C. jejuni colonization in the chicken host, broilers inoculated with C. jejuni 11168, were given B. subtilis PS-216 spore solutions (2.5×106 CFU/mL) in drinking water. Broilers underwent following treatment regiments (FIG. 2): i) No-treatment control group was inoculated with C. jejuni, but not treated with spores (Control); ii) Pre-treatment group was treated with spores for 7 days before broiler inoculation with C. jejuni, to evaluate the ability of B. subtilis PS-216 to prevent C. jejuni colonization of broilers (preventive measure); iii) Continuous treatment group was treated with spores for the entire duration of the experiment (21 days); and iv) Post-treatment group was treated for 8 days after colonization of C. jejuni in chickens, to evaluate the ability of B. subtilis PS-216 to reduce C. jejuni after an already established colonization of broilers (curative measure). Both C. jejuni and Bacillus spores were enumerated in broiler feces during the experiment, and in cecum content after sacrifice.

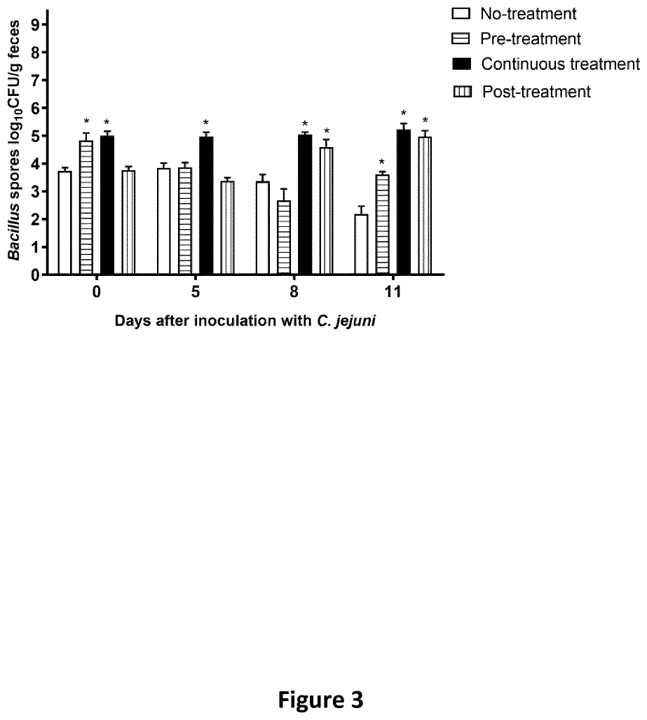

Spore counts in chicken feces were greatly affected by the spore treatment (FIG. 3). Groups receiving spore treatment had significantly higher spore counts compared to untreated groups (p<0.05). Two groups treated before inoculation with C. jejuni (Day 0) had a spore count increase of 1.09 log (Pre-treatment) and 1.26 log (Continuous treatment), compared to the untreated control group. In the Pre-treatment group, 5 days after the treatment was discontinued (Day 5 after C. jejuni inoculation), spore counts decreased to levels of the untreated groups. Spore counts of broilers in the untreated control group decreased with time, but in treated groups these counts stayed comparable for the duration of the treatment. At the last feces sampling (Day 11 after inoculation with C. jejuni) all treated groups (Pre-treatment, Continuous treatment, Post-treatment) showed higher spore counts compared to the untreated control group (increase by 1.57, 3.19 and 2.94 log 10 CFU/g; p<0.05).

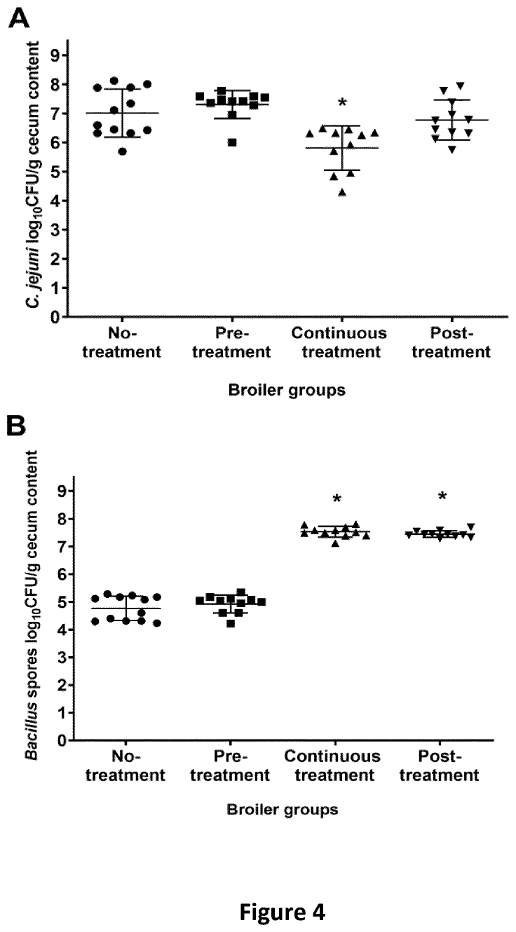

At 21 days of age all chickens were sacrificed, weighted, and tested for C. jejuni and spores. We detected C. jejuni in all chickens, regardless of the treatment regimen (FIG. 4A). A statistically significant decrease in C. jejuni numbers in cecum content (p=0.002), compared to the untreated control group, was detected only in the group continuously treated with B. subtilis PS-216 spores, with a 1.2 log 10 CFU/g average decrease. C. jejuni counts in the Pre-treatment and Post-treatment groups were comparable to the untreated control group.

Spore counts in cecum content of chickens treated with spores shortly before sacrifice (Continuous treatment and Post-treatment) was higher compared to the untreated control group (increase of 2.76 and 2.68 log 10 CFU/g; p<0.01). The 7-day pre-treatment of chickens with spores didn't affect the final spore count, as it was comparable to the untreated control group (FIG. 4B).

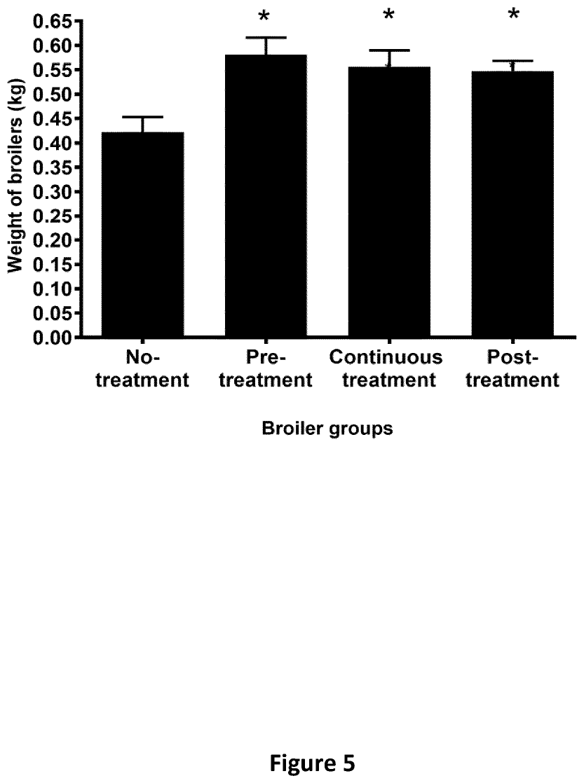

Weight of broilers, at 21 days of age, was significantly higher (p<0.05) in all three groups treated with B. subtilis PS-216 spore solution, compared to the untreated control group (FIG. 5). An average increase of 158 g was detected in the Pre-treatment group, 134 g in the Continuous treatment group, and 124 g in the Post-treatment group.

### Example 2

The ability of B. subtilis PS-216 to reduce enteric and foodborne bacteria was tested in vitro. The conditions used were adjusted as to simulate conditions in the gastrointestinal tract of poultry (i.e. 42° C. and microaerobic conditions). Besides Campylobacter spp., other enteric and foodborne pathogens present major health and economic challenges. Of these, Listeria monocytogenes, Escherichia coli and Salmonella enterica serovars are widespread. Of Salmonella enterica, the top five most commonly reported serovars are S. Enteritidis, S. Typhimurium, monophasic S. Typhimurium, and S. Infantis (EFSA and ECDC, 2018). Thus, we have tested the probiotic potential of B. subtilis PS-216 to reduce these pathogen. The probiotic potential was determined against L. monocytogenes, S. Enteritidis, S. Infantis, and E. coli.

Materials and Methods

L. monocytogenes ZM58, S. Infantis ZM351 and E. coli ZM370, provided by the Laboratory for Food Microbiology (Department of Food Science and Technology, Biotechnical Faculty, University of Ljubljana) were prepared in co-culture with B. subtilis strains PS-216 and T16-10. Co-cultures and control mono-cultures were cultivated in MHB at 42° C. in microaerobic conditions for 24 hours. The concentration of each bacteria in liquid cultures was determined by plating on selective agars. ALOA (Biolife, Milan, Italy) with ALOA enrichment and selective supplement (Biolife, Milan, Italy) was used for L. monocytogenes enumeration, SS (Biolife, Milan, Italy) for S. Enteritidis and S. Infantis, and TBX (Scharlau, Barcelona, Spain) for E. coli.

Results

To determine the influence of B. subtilis PS-216 on other enteric and food pathogens, co-cultures of B. subtilis PS-216 were prepared with L. monocytogenes, S. Enteritidis, S. Infantis, and E. coli. To show the superiority of PS-216 in reduction of enteric and food pathogens in comparison with other B. subtilis, the effect of PS-216 was compared to the effect of B. subtilis T16-10.

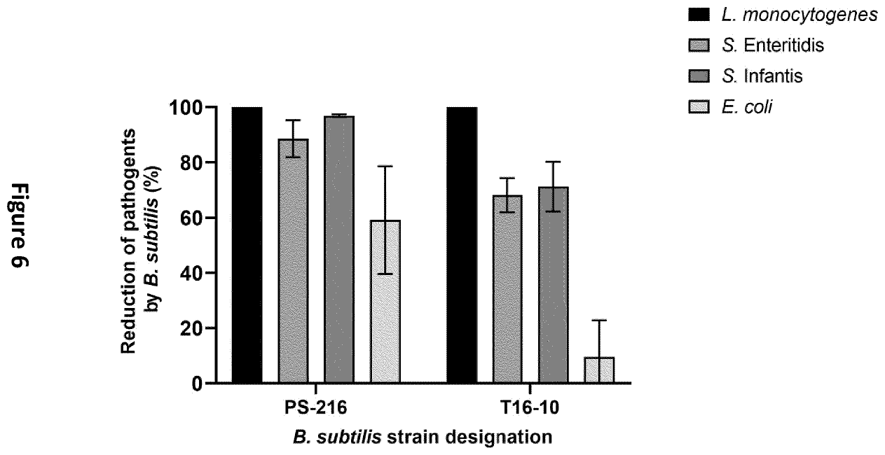

After a 24 h incubation in MHB at 42° C. in microaerobic conditions, a reduction of pathogenic bacteria in co-culture with B. subtilis, was determined. B. subtilis PS-216 reduced L. monocytogenes by >99.99%, S. Enteritidis by 88.6%, S. Infantis by 96.9%, and E. coli by 59.1%. In comparison, B. subtilis T16-10 reduced L. monocytogenes by a similar degree (>99.99%), but showed weaker effect on S. Enteritidis (68.1%), S. Infantis (71.2%), and E. coli (9.6%), as seen in FIG. 6. This shows that B. subtilis PS-216 can reduce enteric and foodborne pathogens in vitro and is superior in its action, compared to other B. subtilis strains. The effect of PS-216 on L. monocytogenes is better than the effect on C. jejuni (99.76%), although the effect on others is somewhat weaker. From these data we conclude that B. subtilis PS-216 can be used against enteric and foodborne pathogens as a probiotic.

### Example 3

Material and Methods

Bacterial Strains and Strain Construction

In mixed-species biofilms experiments a C. jejuni NCTC11168 (WT) (Parkhill et al., 2000) and its derivate tagged with a gfp gene expressed on a plasmid (C. jejuni NCTC11168 cj0046::gfp-knR), (WT-GFP) were used together with a WT B. subtilis PS-216 strain and its derivatives tagged with an mKate fluorescent protein (RFP) linked to a constitutive promotor integrated into two different loci (PS-216 amyE::Phyperspank-mKate2 cmR; PS-216 sacA::P43-mkate2 knR) (WT-RFP1; WT-RFP2).

Growth Conditions

C. jejuni WT strain was sub-cultured form the stock (−80° C.) by cultivation on Karmali agar plates (Oxoid™) with selective supplement SR1607E (Oxoid™), while C. jejuni (WT-GFP) on Müller-Hinton agar (MHA) supplemented with Kanamycin (kn) (50 μg ml⁻¹) for 24 h. After revitalization from −80° C. and prior to experiments, C. jejuni VWT and WT-GFP strains were sub-cultured for additional 24 h on MHA and MHA supplemented with Kn, respectively. All C. jejuni cultivations took place at 42° C. in microaerobic (85% N₂, 5% O₂, 10% CO₂) conditions. All B. subtilis strains were sub-cultured from the stock (−80° C.) by cultivation on MHA or MHA with appropriate antibiotics (Lincomycin (mls) 12.5 μg ml⁻¹+erythromycin 0.5 μg ml⁻¹; spectinomycin (spec) 100 μg ml⁻¹; erythromycin (erm), 20 μg ml⁻¹), (cm, 5 μg ml⁻¹; kn, 50 μg ml⁻¹) (WT RFP_{1,2} strain derivates) for 24 h. After 24 h colonies were sub-cultured on MHA and MHA supplemented with appropriate antibiotics at 28° C. for 24 h in aerobic conditions.

C. jejuni colony counts (CFU/mL) were determined on Karmali agar plates incubated at 42° C. for 24 h in microaerobic conditions. B. subtilis CFU/mL were determined on MHA agar plates incubated at 28° C. for 15-18 h in aerobic conditions, which is selective against the growth of C. jejuni. All B. subtilis-C. jejuni co-cultures experiments were routinely performed in the controlled atmosphere under static microaerobic conditions (85% N₂, 5% O₂, 10% CO₂) at 42° C. in Müller-Hinton broth (MHB).

Mixed Species Biofilms

In order to test the antagonistic activity of B. subtilis strain PS-216 against C. jejuni in mixed species biofilm cultures we first diluted the overnight cultures of each species to OD₆₀₀=0.1. and used 0.1% inoculum (each) in 5 mL of MHB. This approach assured 1:10 ratio of B. subtilis versus C. jejuni with ˜4.3 log₁₀ CFU/mL and ˜5.3 log 10 CFU/mL) respectively. Before each sampling cultures were vortexed, sampled and then incubated statically until the next disruptive sampling. Monocultures of B. subtilis and C. jejuni were used as controls (0.1% inoculum). Both species were mixed at 1:10 ration in MHB as described above and incubated at microaerobic conditions, at 42° C. and CFU/mL determined at time 0 h and after 24 h. Biofilms were disrupted by vortexing and pipetting before plating for CFU.

Trans-Well Co-Culture

In order to test whether B. subtilis produces diffusible substances, which inhibit the growth of C. jejuni, both species were co-cultured in a system that allowed the exchange of molecules but not direct contact between cells. Each well on the 12-well plate (Cellstar, Greiner, bio-one) contained two chambers separated by a 0.1 μm pore size membrane. Fist, C. jejuni was inoculated into the well containing 3 mL of MHB, after which an inlay (2 mL) containing B. subtilis was submerged into the well containing C. jejuni. Inoculum of B. subtilis and C. jejuni was prepared as described above. Monocultures of B. subtilis and C. jejuni were used as controls (0.1% inoculum). C. jejuni culture was inoculated into the well after which an inlay containing only MHB was submerged, and B. subtilis was inoculated into the inlay submerged into well containing only MHB. The CFU/mL of B. subtilis and C. jejuni mono- and co-cultures was determined at 0 h and 24 h.

Adhesion to an Inert Surface

The adhesion capability of B. subtilis PS-216 and C. jejuni WT was determined by the number of adhered cells (CFU/mL) to the inert polystyrene surface. Inocula of B. subtilis and C. jejuni were prepared from overnight cultures by diluting the culture to OD₆₀₀=0.1. Cells of each species were mixed in 1:1 ratio (in 100 μL) with the final concentration of both strains ˜6 log₁₀ CFU/mL in each well of the 96-well microtiter plate (Nunc, Roskilde, Denmark). Monocultures of B. subtilis and C. jejuni at the same initial cell numbers were used as controls. Following the 24 h incubation in microaerobic conditions at 42° C., planktonic/unattached cells were removed by repeated (3×) rinsing of the polystyrene surface with 100 μL sterile phosphate-buffer saline (PBS). Attached cells were removed by sonicating the plates (room temperature, 10 min; frequency, 28 kHz; power 300 W; Iskra Pio, Šentjernej, Slovenia) and resuspended in 100 μL of PBS.

Disruption of Pre-Established Biofilm

An ability of B. subtilis to disrupt the biofilm of C. jejuni was tested by first allowing C. jejuni to form a biofilm. C. jejuni VWT was inoculated at ˜6 log 10 CFU/mL, (OD₆₀₀=0.1) in fresh MHB was, incubated for 26 h at 42° C. in microaerobic conditions and then B. subtilis—PS-216 culture (˜4.2 log 10 CFU/mL) was added. This experiment was performed in 6-well polystyrene microtiter plates (volume 5 mL) (TPP, Switzerland) for CFU/mL counts. Colony counts (CFU/mL) were determined for C. jejuni at the beginning of incubation and for both species at to (the point when B. subtilis was added), at t₁₂ (12 h after co-incubation) and t₂₂ (22 h after co-incubation). In order to visualize the effect of B. subtilis on preestablished C. jejuni biofilm we co-cultured fluorescently labelled strains, C. jejuni WT-GFP (green) and B. subtilis WT-RFP₂ (red) in 96-well microtiter plate with glass bottom (TPP, Switzerland) at 42° C. and biofilms visualized by confocal laser scanning microscopy (CLSM) at indicated times.

Spatial Distribution of B. subtilis and C. jejuni Cells in Co-Culture

Mixed and mono species biofilms of B. subtilis PS-216 WT-RFP₁ and C. jejuni WT-GFP were grown in MHB in 96-well microtiter plate (TPP, Switzerland) at microaerobic conditions and 42° C. Strains in co-culture were mixed in 1:1 ratio (in 100 μL) with the final concentration of both strains ˜6 log 10 CFU/mL and with the same number of cells (6 log 10 CFU/ml) for monocultures. After 24 h of incubation, we performed biofilms imaging by confocal laser scanning microscopy (CLSM). For colony counts of both species we cultured biofilms in 5 mL of MHB (6 well microtiter plates) and sampled the pellicle and the broth below the pellicle at 0 h and 24 h. Before plating CFU/ml pellicles were rinsed by repeated 3×immersion in the sterile PBS, and repetitively vortexed before and after sonication (room temperature, 2×10 min; frequency, 28 kHz; power 300 W; Iskra Pio, Šentjernej, Slovenia). B. subtilis and C. jejuni monocultures were used as controls.

Light Field and Confocal Laser Scanning Microscopy (CLSM)

Spatial distribution and structural properties of B. subtilis and C. jejuni biofilms in mono and co-culture were investigated using the inverted confocal laser scanning (CLSM) microscope Axiovsion Z1, LSM800 (Zeiss, Germany). Biofilms were grown as described above and imaging performed directly in microtiter wells. We used C. jejuni strains tagged with GFP (green) and B. subtilis tagged with RFP1 (red). Fluorescent reporters excitation for GFP was performed at 488 nm with an argon laser, and the emitted fluorescence was recorded 400-580 nm. Excitation of the red fluorescence protein RFP (mKate) was performed at 561 nm, while the emitted fluorescence was recorded at 580-700 nm. The laser intensities and GaAsP detector gain were 4%, 800 V and 4.5%, 650 V for mKate (RFP) and GFP channel, respectively. Pinhole size was 55 μm. To generate images of the biofilms a minimum of of 124 Z-image series with a 10 μm size stack for each biological sample. The acquired images were typically 1024×1024 pixels size with 16 bit colour depth and microtiter wells were scanned using 20×/0.4 N.A. objective. The bright-field images were acquired using Axiocam MRm rev. 3 (Zeiss) camera and HAL 100 light source (Zeiss). Zen 2.3 Software (Carl Zeiss) was used for acquiring and image visualization. The noise on acquired CLSM images was reduced by applying single pixel filter (threshold=1.5).

Statistical Analysis

In order to evaluate the influence of biofilm co-cultivation on the growth of B. subtilis and C. jejuni, statistical significance was assessed by the Two-Sample t-test using raw data. Probability values smaller than 0.05 (P<0.05) were considered statistically significant (equal variance not assumed—Welch correction). Three biological and up to three technical replicas were used for all experiments. The data are displayed as mean values±the standard deviation of the mean value. The entire analysis was performed using OriginPro 2020 (OriginLab Corporation, Northampton, MA USA).

### Results

PS-216 Inhibits the Growth of C. jejuni and its Adhesion to a Polystyrene Surface

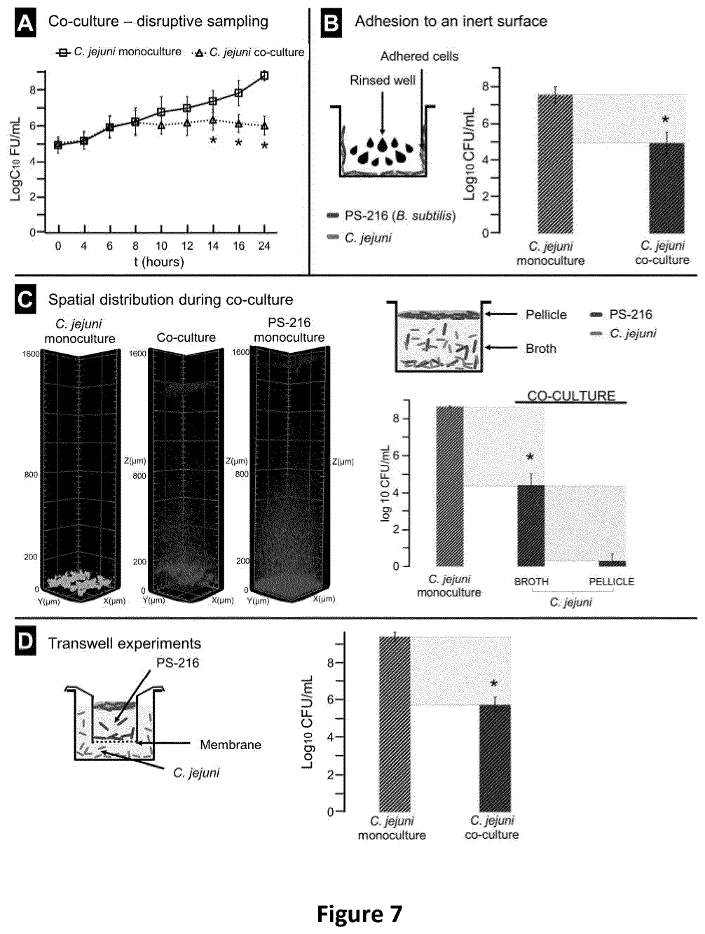

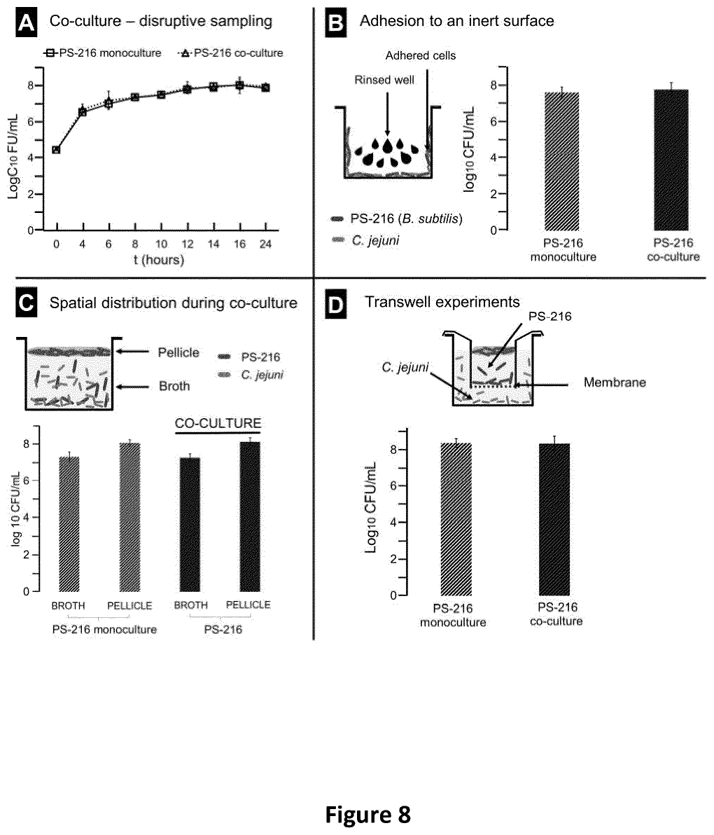

C. jejuni represents a major health threat in the general human population. A key issue during food production is transmission by abiotic surfaces, where the formation of biofilms can assist in C. jejuni growth and survival. Here we investigate the potential of B. subtilis PS-216 to antagonize C. jejuni growth in static co-culture inoculated at 1:10 ratio (B. subtilis:C. jejuni) and CFU/mL of each species determined at specific times by plating the samples on selective media. B. subtilis significantly inhibited C. jejuni at 14 h (P₍₁₄₎=0.0046), 16 h (P₍₁₆₎=0.009) and 24 h of co-incubation (p₍₂₄₎=4 E-4). The CFU/mL of C. jejuni after 24 h incubation at 42° C. in co-culture with B. subtilis was significantly lower (6 log₁₀ CFU/mL) than the CFU of C. jejuni monoculture (8.8 log₁₀ CFU/mL) (P₍₂₄ₕ₎=4 10⁻⁴), with the 2.8 log₁₀ reduction in CFU/mL of C. jejuni cells in the presence of B. subtilis. (FIG. 7A). At earlier time points 10 h (P₍₁₀₎=0.22) and 12 h (P₍₁₂₎=0.14) of co-incubation inhibition was visible but not significant. C. jejuni did not affect the growth of B. subtilis PS-216 with (P₍₁₀₎=0.33; P₍₁₂₎=0.64; P₍₁₄₎=0.55; P₍₁₅₎=0.87; P₍₂₄₎=0.5) (FIG. 8A).

C. jejuni is known to adhere to abiotic surfaces and form robust biofilms (Bronnec et al., 2016). Therefore, we tested whether B. subtilis reduces the adhesion of C. jejuni to polystyrene surfaces. We quantified attached cells after B. subtilis and C. jejuni mixed at 1:1 ratio were grown 42° C. for 24 h. The number (CFU/ml) of adhered C. jejuni cells in mixed species biofilm was significantly lower (4.9 log₁₀ CFU/mL) than in monoculture (7.4 log₁₀ CFB CFU/mL) (P₍₂₄₎=0.002). The presence of B. subtilis, therefore, reduced the CFU/mL of C. jejuni by 2.4 log₁₀ (FIG. 7B). In contrast, CFU/mL of adhered B. subtilis PS-216 in monoculture and co-culture were comparable (P₍₂₄₎=0.54) (FIG. 8B).

C. jejuni and B. subtilis Biofilms Segregate in Co-Culture

B. subtilis is a ubiquitous bacterium that occurs regularly in poultry GIT or in the poultry environment (Humphrey et al., 2014), where it can potentially interact and interfere with C. jejuni. However, the C. jejuni-B. subtilis interspecies interactions in biofilms are poorly understood. To further investigate mixed species biofilms we incubated fluorescently tagged C. jejuni (WT-GFP) in the presence and absence of fluorescently tagged B. subtilis PS-216 (WT-RFP1) grown for 24 h at 42° C. in 96-well microtiter plates. Confocal scanning microscopy that C. jejuni predominantly occupied the bottom of the well in mono cultures forming submerged cell aggregates with characteristic finger-like structures (C. jejuni submerged biofilm) (FIG. 7C). However, when grown with B. subtilis, the biofilm formation of C. jejuni was not visible. B. subtilis PS-216 biofilm, on the other hand, was not affected by the presence of C. jejuni. B. subtilis cells formed biofilm at the bottom of the well (B. subtilis submerged biofilm) and at the air-liquid interface (B. subtilis pellicle) (FIG. 7C) regardless of C. jejuni.

We also quantified both species in the pellicle and below the pellicle by CFU/mL. After 24 h of static growth in MHB we sampled the pellicle and the submerged area (planktonic cells in the medium and the submerged biofilm) for CFU/mL. After 24 h incubation in co-culture with B. subtilis C. jejuni dropped for 4.2 log₁₀. In monoculture C. jejuni grew to 8.6 log₁₀ CFU/mL but in co-culture only to 4.4 log₁₀ CFU/mL (P₍₂₄ₕ₎=7 10⁻⁶)(FIG. 1C). The pellicle mostly consisted of B. subtilis (7.3 log₁₀ CFU/mL) (FIG. 8C) with only 0.3 log₁₀ CFU/mL of C. jejuni (FIG. 7C). The B. subtilis growth remained stable, with no difference in CFU/mL of B. subtilis PS-216 in the monoculture broth versus in co-culture with C. jejuni (P₍₂₄₎=0.72) (FIG. 8C: Also, we did not detect any difference in the pellicle CFUs of B. subtilis PS-216 in monoculture and co-culture with C. jejuni (P₍₂₄₎=0.51) (FIG. 8C).

Cell to Cell Contact is not Necessary for Anti-Campylobacter Activity of PS-216 Strain

Killing of the attacked bacterial species may be due to diffusible factors or contact dependent killing (add reference Kalamara et al., 2018). To test whether cell-cell contact between B. subtilis and C. jejuni is required for the inhibition of the latter, we physically separated the two species in a trans-well experiment using a 0.1 μm pore size membrane between two incubation chambers, each containing one species. The lower chamber contained C. jejuni species, after which an inlay of B. subtilis was submerged into the C. jejuni chamber. Our results show, that inhibition of C. jejuni by B. subtilis PS-216 is not cell-contact dependent, since the CFU/mL of C. jejuni after 24 h incubation in co-culture with B. subtilis was significantly lower (5.7 log₁₀ CFU/mL) than the CFU/mL of C. jejuni monoculture (9.4 log₁₀ CFU/mL) (P₍₂₄ₕ₎=7.6 10⁻⁹), with 3.7 log₁₀ reduction CFU/mL of C. jejuni cells in the presence of B. subtilis. (FIG. 7D). On the other hand, C. jejuni again did not affect the growth of B. subtilis, because no difference in growth of B. subtilis PS-216 was observed in monoculture compared to when B. subtilis was co-cultured with C. jejuni (P₍₂₄₎=0.59) (FIG. 8D).

B subtilis PS-216 Affects the Preestablished C. jejuni Submerged Biofilm

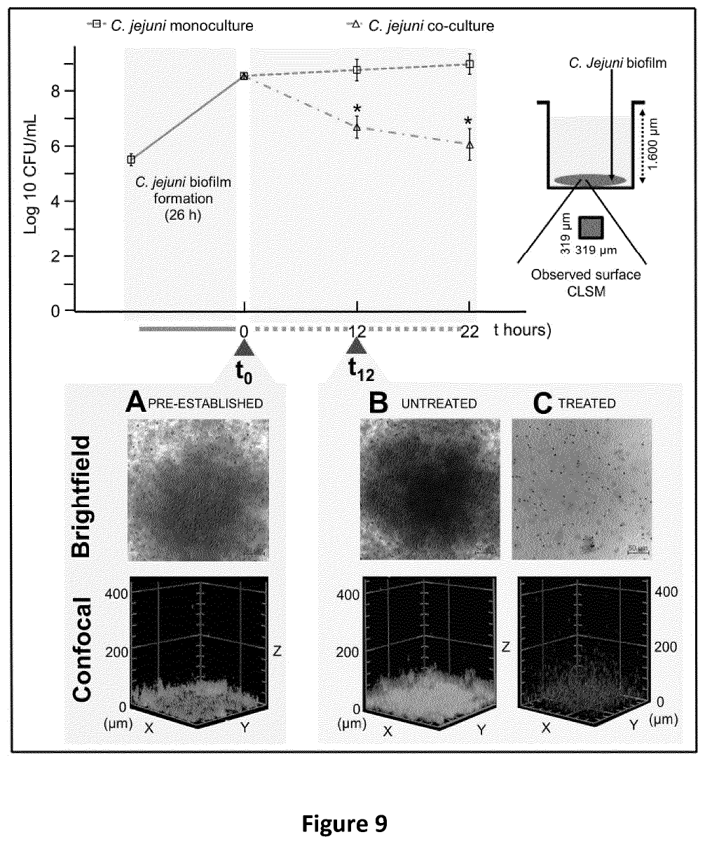

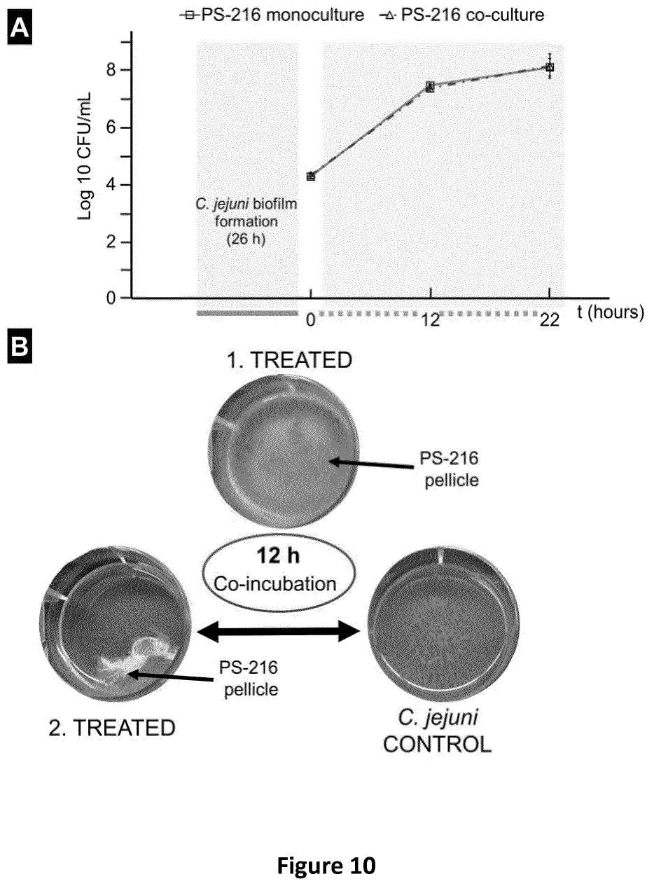

To test whether B. subtilis PS-216 is capable to disrupt the preestablished C. jejuni biofilm we introduced the B. subtilis PS-216 to 26 h old C. jejuni culture with a preestablished submerged biofilm (to) in a 1:10000 (B. subtilis:C. jejuni) ratio. Using CLSM and bright field microscopy, the C. jejuni submerged biofilm was no longer visible after 12 h of B. subtilis-C. jejuni co-incubation (t₁₂) (FIG. 9). Similarly, B. subtilis PS-216 significantly reduced the CFU/mL of C. jejuni at t₁₂ (P₍₁₂ₕ₎>=5 10⁻⁸) and t₂₂ (P₍₂₂ₕ₎=3.4 10⁻⁶) by 2.1 log₁₀ CFU/mL and 2.8 log₁₀ CFU/mL, respectively (FIG. 9). On the other hand, no inhibition of B. subtilis by C. jejuni was observed at t₁₂ (P₍₁₂ₕ₎=0.045) and t₂₂ (P₍₂₂ₕ₎=0.85) (FIG. 10A). Taken together, our results show that B. subtilis PS-216 is able to disintegrate the preestablished C. jejuni biofilm in as little as 12 h of co-incubation in microaerobic conditions, leaving no visible aggregates and finger-like structures, characteristic for C. jejuni biofilms (FIG. 10B).

### LIST OF CERTAIN REFERENCES CITED IN THE DESCRIPTION

Kaakoush N O, Castaño-Rodriguez N, Mitchell H M, Man S M. 2015. Global Epidemiology of Campylobacter Infection. Clinical Microbiology Reviews 28:687-720.
Nachamkin, Irving, Ban Mishu Allos, and Tony Ho. 1998. “Campylobacter Species and Guillain-Barré Syndrome.” Clinical Microbiology Reviews 11 (3): 555-67. https://www.ncbi.nlm.nih.gov/pmc/articles/PMC88896/.
EFSA and ECDC. 2018. “European Food Safety Authority and European Centre for Disease Prevention and Control. The European Union Summary Report on Trends and Sources of Zoonoses, Zoonotic Agents and Food-Borne Outbreaks in 2017.” EFSA Journal 16 (12): e05500. https://doi.org/10.2903/j.efsa.2018.5500.
BIOHAZ. 2011. “EFSA Panel on Biological Hazards. Scientific Opinion on Campylobacter in Broiler Meat Production: Control Options and Performance Objectives and/or Targets at Different Stages of the Food Chain.” EFSA Journal 9 (4). https://doi.org/10.2903/j.efsa.2011.2105.
Anonymous. 2019. The European Union One Health 2018 Zoonoses Report. EFSA Journal 17:e05926.
Berndtson, E., M. L. Danielsson-Tham, and A. Engvall. 1996. “Campylobacter Incidence on a Chicken Farm and the Spread of Campylobacter during the Slaughter Process.” International Journal of Food Microbiology 32 (1-2): 35-47. https://doi.org/10.1016/0168-1605(96)01102-6.
Sahin, Orhan, Issmat I. Kassem, Zhangqi Shen, Jun Lin, Gireesh Rajashekara, and Qijing Zhang. 2015. “Campylobacter in Poultry: Ecology and Potential Interventions.” Avian Diseases 59 (2): 185-200. https://doi.org/10.1637/11072-032315-Review.
Meunier, M., M. Guyard-Nicodeme, D. Dory, and M. Chemaly. 2016. “Control Strategies against Campylobacter at the Poultry Production Level: Biosecurity Measures, Feed Additives and Vaccination.” Journal of Applied Microbiology 120 (5): 1139-73. https://doi.org/10.1111/jam.12986.
Dogan, Onay Burak, Jennifer Clark, Fabio Mattos, and Bing Wang. 2019. “A Quantitative Microbial Risk Assessment Model of Campylobacter in Broiler Chickens: Evaluating Processing Interventions.” Food Control 100 (June): 97-110. https://doi.org/10.1016/j.foodcont.2019.01.003.
Alagawany, Mahmoud, Mohamed E. Abd El-Hack, Mayada R. Farag, Swati Sachan, Kumaragurubaran Karthik, and Kuldeep Dhama. 2018. “The Use of Probiotics as Eco-Friendly Alternatives for Antibiotics in Poultry Nutrition.” Environmental Science and Pollution Research 25 (11): 10611-18. https://doi.org/10.1007/s11356-018-1687-x.
Hong, Huynh A., Le Hong Duc, and Simon M. Cutting. 2005. “The Use of Bacterial Spore Formers as Probiotics.” FEMS Microbiology Reviews 29 (4): 813-35. https://doi.org/10.1016/j.femsre.2004.12.001.
Lutful Kabir, S. M. 2009. “The Role of Probiotics in the Poultry Industry.” International Journal of Molecular Sciences 10 (8): 3531-46. https://doi.org/10.3390/ijms10083531.
Fritts, C. A., J. H. Kersey, M. A. Motl, E. C. Kroger, F. Yan, J. Si, Q. Jiang, M. M. Campos, A. L. Waldroup, and P. W. Waldroup. 2000. “Bacillus Subtilis C-3102 (Calsporin) Improves Live Performance and Microbiological Status of Broiler Chickens.” Journal of Applied Poultry Research 9 (2): 149-55. https://doi.org/10.1093/japr/9.2.149.
Hayashi, Ricardo Mitsuo, Mariana Camargo Lourengo, Antônio Leonardo Kraieski, Raquel Bighetti Araujo, Ricardo Gonzalez-Esquerra, Eduardo Leonardecz, Anderson Ferreira da Cunha, Marcelo Falsarella Carazzolle, Paulo Sérgio Monzani, and Elizabeth Santin. 2018. “Effect of Feeding Bacillus Subtilis Spores to Broilers Challenged with Salmonella Enterica Serovar Heidelberg Brazilian Strain UFPR1 on Performance, Immune Response, and Gut Health.” Frontiers in Veterinary Science 5 (February). https://doi.org/10.3389/fvets.2018.00013.
Hmani, Houda, Lobna Daoud, Mouna Jlidi, Karim Jalleli, Manel Ben Ali, Adel Hadj Brahim, Mansour Bargui, Alaeddine Dammak, and Mamdouh Ben Ali. 2017. “A Bacillus Subtilis Strain as Probiotic in Poultry: Selection Based on in Vitro Functional Properties and Enzymatic Potentialities.” Journal of Industrial Microbiology & Biotechnology 44 (8): 1157-66. https://doi.org/10.1007/s10295-017-1944-x.
Jayaraman, Sathishkumar, Gokila Thangavel, Hannah Kurian, Ravichandran Mani, Rajalekshmi Mukkalil, and Haridasan Chirakkal. 2013. “Bacillus Subtilis PB6 Improves Intestinal Health of Broiler Chickens Challenged with Clostridium Perfringens-Induced Necrotic Enteritis.” Poultry Science 92 (2): 370-74. https://doi.org/10.3382/ps.2012-02528.
Park, Jae-Hong, Yong-Min Kim, Dae-Kyung Kang, and In-Ho Kim. 2017. “Effect of Dietary Bacillus Subtilis C14 and RX7 Strains on Growth Performance, Blood Parameter, and Intestinal Microbiota in Broiler Chickens Challenged with Salmonella Gallinarum.” The Journal of Poultry Science 54 (3): 236-41. https://doi.org/10.2141/jpsa.0160078.
Zhou, Xianjian, Erhui Jin, Shenghe Li, Chenfang Wang, Enmei Qiao, and Guozhong Wu. 2015. “Effects of dietary supplementation of probiotics (Bacillus subtilis, Bacillus licheniformis, and Bacillus natto) on broiler muscle development and meat quality.” Turkish Journal of Veterinary and Animal Sciences 39 (2): 203-10. https://doi.org/10.3906/vet-1406-67.
Saint-Cyr, Manuel J., Muriel Guyard-Nicodeme, Soumaya Messaoudi, Marianne Chemaly, Jean-Michel Cappelier, Xavier Dousset, and Nabila Haddad. 2016. “Recent Advances in Screening of Anti-Campylobacter Activity in Probiotics for Use in Poultry.” Frontiers in Microbiology 7. https://doi.org/10.3389/fmicb.2016.00553.
Kovač, Jasna, Blaž Stessl, Neža Čadež, Igor Gruntar, Matej Cimerman, Katarina Stingl, Marija Lušicky, Matjaž Ocepek, Martin Wagner, and Sonja Smole Možina. 2018. “Population Structure and Attribution of Human Clinical Campylobacter Jejuni Isolates from Central Europe to Livestock and Environmental Sources.” Zoonoses and Public Health 65 (1): 51-58. https://doi.org/0.1111/zph.12366.
Kovač, Jasna, Katarina Šimunović, Zuowei Wu, Anja Klančnik, Franz Bucar, Qijing Zhang, and Sonja Smole Možina. 2015. “Antibiotic Resistance Modulation and Modes of Action of (−)-α-Pinene in Campylobacter Jejuni.” PLOS ONE 10 (4): e0122871. https://doi.org/10.1371/journal.pone.0122871.
Warriner, K., and W. M. Waites. 1999. “Enhanced Sporulation in Bacillus Subtilis Grown on Medium Containing Glucose:Ribose.” Letters in Applied Microbiology 29 (2): 97-102. https://doi.org/10.1046/j.1365-2672.1999.00593.x.
EFSA. 2012. “Guidance on the Assessment of Bacterial Susceptibility to Antimicrobials of Human and Veterinary Importance.” EFSA Journal 10 (6): 2740. https://doi.org/10.2903/j.efsa.2012.2740.
Barbosa, Teresa M., Cleudia R. Serra, Roberto M. La Ragione, Martin J. Woodward, and Adriano O. Henriques. 2005. “Screening for Bacillus Isolates in the Broiler Gastrointestinal Tract.” Applied and Environmental Microbiology 71 (2): 968-78. https://doi.org/10.1128/AEM. 71.2.968-978.2005.
Mingmongkolchai, S., and W. Panbangred. 2018. “Bacillus Probiotics: An Alternative to Antibiotics for Livestock Production.” Journal of Applied Microbiology 124 (6): 1334-46. https://doi.org/10.1111/jam.13690.
Štefanič, Polonca, and Ines Mandič-Mulec. 2009. “Social Interactions and Distribution of Bacillus Subtilis Pherotypes at Microscale.” Journal of Bacteriology 191 (6): 1756-64. https://doi.org/10.1128/JB.01290-08.
Parkhill, J., B. W. Wren, K. Mungall, J. M. Ketley, C. Churcher, D. Basham, T. Chillingworth, et al. 2000. “The Genome Sequence of the Food-Borne Pathogen Campylobacter Jejuni Reveals Hypervariable Sequences.” Nature 403 (6770): 665-68. https://doi.org/10.1038/35001088.
Bronnec, Vicky, Hana Turoňová, Agnès Bouju, Stéphane Cruveiller, Ramila Rodrigues, Katerina Demnerova, Odile Tresse, Nabila Haddad, and Monique Zagorec. 2016. “Adhesion, Biofilm Formation, and Genomic Features of Campylobacter Jejuni Bf, an Atypical Strain Able to Grow under Aerobic Conditions.” Frontiers in Microbiology 7. https://doi.org/10.3389/fmicb.2016.01002.
Humphrey, Suzanne, Gemma Chaloner, Kirsty Kemmett, Nicola Davidson, Nicola Williams, Anja Kipar, Tom Humphrey, and Paul Wigley. 2014. “Campylobacter Jejuni Is Not Merely a Commensal in Commercial Broiler Chickens and Affects Bird Welfare.” MBio 5 (4). https://doi.org/10.1128/mBio.01364-14.

1. A method for enhancing the growth of a non-human animal, such as poultry animal, comprising administering an effective amount of the Bacillus subtilis strain PS-216, or a derivative thereof having all of the identifying characteristics of B. subtilis PS-216, to the non-human animal.

2. A method for increasing the weight of a non-human animal, such as poultry animal, comprising administering an effective amount of the B. subtilis strain PS-216, or a derivative thereof having all of the identifying characteristics of B. subtilis PS-216, to the non-human animal.

3. The method according to claim 1 or 2, wherein the non-human animal is a productive animal.

4. The method according to claim 3, wherein the productive animal is selected from the group consisting of pig, chicken, duck, goose, turkey, cow, sheep and goat.

5. The method according to any one of claims 1 to 4, wherein the non-human animal is a poultry animal.

6. The method according to claim 5, wherein the poultry animal is a productive poultry animal.

7. The method according to claim 5 or 6, wherein the poultry animal is selected from the group consisting of: chicken, duck, goose, turkey, guinea fowl and pigeon.

8. The method according to claim 5 or 6, wherein the poultry animal is a chicken.

9. The method according to any one of claims 1 to 8, wherein the non-human animal is under four weeks old.

10. The method according to any one of claims 1 to 9, wherein the non-human animal is between 1 and 21 days old.

11. The method according to any one of claims 1 to 10, wherein the non-human animal is between 1 and 14 days old.

12. The method according to any one of claims 1 to 11, wherein the strain is administered in the form of spores, cells, vegetative cells or a dried cell mass.

13. The method according to any one of claims 1 to 12, wherein the strain is administered in the form of spores.

14. The method according to any one of claims 1 to 13, wherein the strain is administered at a bacterial load of at least about 1×10² colony forming units (CFU).

15. The method according to any one of claims 1 to 14, wherein the strain is administered at a bacterial load in a range of from about 1×10² to about 1×10¹⁴ colony forming units.

16. The method according to any one of claims 1 to 15, wherein the strain is administered in an aqueous solution.

17. The method according to any one of claims 1 to 15, wherein the strain is administered as a dry feed.

18. The method according to any one of claims 1 to 15, wherein the strain is administered as part of the feed composition according to any one of claims 20 to 25.

19. The method according to any one of claims 1 to 18, wherein the strain is administered perorally.

20. A feed or food composition containing the B. subtilis strain PS-216, or a derivative thereof having all of the identifying characteristics of B. subtilis PS-216, and at least one further feed or food ingredient.

21. The feed or food composition according to claim 20, wherein the feed or food ingredient is selected from proteins, carbohydrates, fats, fibers, further probiotics, prebiotics, enzymes, vitamins, minerals, amino acids, and combinations thereof.

22. The feed or food composition according to claim 20 or 21, wherein the strain is in the form of spores, cells, vegetative cells or a dried cell mass.

23. The feed or food composition according to any one of claims 20 to 22, wherein the strain is in the form of spores.

24. The feed or food composition according to any one of claims 20 to 23, containing at least about 1×10³ colony forming units (CFU) of the strain/kg of composition.

25. The feed or food composition according to any one of claims 20 to 24, containing from about 1×10³ to about 1×10¹⁴ colony forming units of the strain/kg of composition.

26. A method of preparing a feed or food composition according to any one of claims 20 to 25, comprising mixing the B. subtilis strain PS-216, or a derivative thereof having all of the identifying characteristics of B. subtilis PS-216, with at least one feed ingredient.

27. Bacillus subtilis strain PS-216, or a derivative thereof having all of the identifying characteristics of B. subtilis PS-216, for use in inhibiting, reducing or preventing the colonization of an enteropathogenic and/or foodborne pathogenic bacterium, such as C. jejuni, in a subject.

28. B. subtilis strain for use according to claim 27, wherein the enteropathogenic and/or foodborne pathogenic bacterium is a gram-negative bacterium.

29. B. subtilis strain for use according to claim 27 or 28, wherein the enteropathogenic and/or foodborne pathogenic bacterium is a bacterium of the family Campylobacteraceae or Enterobacteriaceae.

30. B. subtilis strain for use according to any one of claims 27 to 29, wherein the enteropathogenic and/or foodborne pathogenic bacterium is a bacterium of the family Campylobacteraceae.

31. B. subtilis strain for use according to any one of claims 27 to 30, wherein the enteropathogenic and/or foodborne pathogenic bacterium is a bacterium of the genus Campylobacter.

32. B. subtilis strain for use according to any one of claims 27 to 31, wherein the enteropathogenic and/or foodborne pathogenic bacterium is a bacterium selected from the group consisting of: C. jejuni, C. coli, C. concisus, C. fetus, C. hyoilei, C. helveticus, C. hyointestinalis, C. lari, C. mucosalis, C. sputorum and C. upsaliensis.

33. B. subtilis strain for use according to any one of claims 27 to 32, wherein the enteropathogenic and/or foodborne pathogenic bacterium is a bacterium of the species C. jejuni or C. coli.

34. B. subtilis strain for use according to any one of claims 27 to 33, wherein the enteropathogenic and/or foodborne pathogenic bacterium is a bacterium of the species C. jejuni.

35. B. subtilis strain for use according to any one of claims 27 to 29, wherein the enteropathogenic and/or foodborne pathogenic bacterium is a bacterium of the family Enterobacteriaceae.

36. B. subtilis strain for use according to any one of claims 27 to 29, wherein the enteropathogenic and/or foodborne pathogenic bacterium is a bacterium of the genus Listeria, Escherichia, Yersinia, Vibrio, Salmonella or Shigella.

37. B. subtilis strain for use according to any one of claims 27 to 29, wherein the enteropathogenic and/or foodborne pathogenic bacterium is a bacterium selected from the group consisting of: Listeria monocytogenes, Yersinia enterocolitica, Vibrio parachaemoliticus, Escherichia coli, Salmonella enterica, including Salmonella Enteritidis and Salmonella Infantis, and Shigella dysenteriae.

38. B. subtilis strain PS-216, or a derivative thereof having all of the identifying characteristics of B. subtilis PS-216, for use in improving the health status, such as the gut health status, of a subject.

39. B. subtilis strain for use according to any one of claims 27 to 38, wherein the subject is a non-human animal.

40. B. subtilis strain for use according to claim 39, wherein the non-human animal is a productive animal.

41. B. subtilis strain for use according to claim 40, wherein the productive animal is selected from the group consisting of pig, chicken, duck, goose, turkey, cow, sheep and goat.

42. B. subtilis strain for use according to any one of claims 39 to 41, wherein the non-human animal is a poultry animal.

43. B. subtilis strain for use according to claim 42, wherein the poultry animal is a productive poultry animal.

44. B. subtilis strain for use according to claim 42 or 43, wherein the poultry animal is selected from the group consisting of: chicken, duck, goose, turkey, guinea fowl and pigeon.

45. B. subtilis strain for use according to claim 42 or 43, wherein the poultry animal is a chicken.

46. B. subtilis strain for use according to any one of claims 27 to 38, wherein the subject is a human.

47. B. subtilis strain for use according to any one of claims 27 to 46, wherein the strain is in the form of spores, cells, vegetative cells or a dried cell mass.

48. B. subtilis strain for use according to any one of claims 27 to 47, wherein the strain is administered in the form of spores.

49. B. subtilis strain for use according to any one of claims 27 to 48, wherein the strain is administered at a bacterial load of at least about 1×10² colony forming units (CFU).

50. B. subtilis strain for use according to any one of claims 27 to 49, wherein the strain is administered at a bacterial load in a range of from about 1×10² to about 1×10¹⁴ colony forming units.

51. B. subtilis strain for use according to any one of claims 27 to 50, wherein the strain is administered perorally.

52. A probiotic composition comprising the B. subtilis strain PS-216, or a derivative thereof having all of the identifying characteristics of B. subtilis PS-216, and at least one excipient, such as at least one pharmaceutically or agriculturally acceptable excipient.

53. The probiotic composition according to claim 52, wherein the strain is in the form of spores, cells, vegetative cells or a dried cell mass.

54. The probiotic composition according to claim 52 or 53, wherein the strain is in the form of spores.

55. The probiotic composition according to any one of claims 52 to 54, containing at least about 1×10³ colony forming units (CFU) of the strain/kg of composition.

56. The probiotic composition according to any one of claims 52 to 55 for use in the treatment and/or prevention of a microbial infection or colonization by an enteropathogenic and/or foodborne pathogenic bacterium, such as C. jejuni, in a subject.
